(19) United States
(12) Patent Application Publication
(10) Pub. No.: US 2003/0045489 A1
(43) Pub. Date: Mar. 6, 2003

(54) Methods for modulating angiogenesis

(76) Inventors: John E. Murphy, Oakland, CA (US); Jianmin Chen, Concord, CA (US); Nathalle Dubois-Stringfellow, Berkeley, CA (US); Dadong Chen, Evanston, IL (US)

Correspondence Address: BAYER CORPORATION, 800 DWIGHT WAY, P.O. BOX 1986, BERKELEY, CA, 94701-1986, US

(21) Appl. No.: 09/996,324

(22) Filed: Nov. 21, 2001

Related U.S. Application Data
(60) Provisional application No. 60/252,748, filed on Nov. 22, 2000.

(51) Int. Cl. A61K 48/00; A61K 38/18

(52) U.S. Cl. 514/44; 514/12

(57) ABSTRACT

The present invention provides methods for modulating angiogenesis by administering anti-angiogenic FGF-19 polypeptides to a subject. Methods of modulating angiogenesis by administering an anti-angiogenic FGF-19 nucleic acid are also provided.

### BACKGROUND OF THE INVENTION

Angiogenesis is generally thought to be tightly regulated by growth factors and other ligands. Angiogenesis, and the concurrent tissue development and regeneration, depends on the tightly controlled processes of endothelial cell proliferation, migration, differentiation and survival. Both stimulator and inhibitor ligands appear to interact, directly or indirectly, with cellular receptors during these processes. Angiogenesis begins with the erosion of the basement membrane by enzymes released by endothelial cells and leukocytes. The endothelial cells, which line the lumen of blood vessels, then protrude through the basement membrane. Angiogenic stimulators induce endothelial cells to migrate through the eroded basement membrane. The migrating cells then form a “sprout” off the parent blood vessel, where the endothelial cells undergo mitosis and proliferate. The endothelial sprouts merge with each other to form capillary loops, creating the new blood vessel.

The factors involved in endothelial cell regulation are beginning to be elucidated. In particular, the fibroblast growth factors (FGFs), originally identified as stimulators of fibroblast proliferation, have been found to have important and diverse biological activities, including angiogenesis, cell differentiation, limb and organ development, wound repair, hair growth, neuronal development, and vascular function (Burgess, W. H., and Maciaq, T., Annu. Rev. Biochem. 58:575-606 (1989)). Fibroblast growth factors are a family of proteins characterized by binding to heparin and are, therefore, also called heparin binding growth factors (HBGFs). Expression of different members of this protein family is found in various tissues and under particular temporal and spatial control. These proteins are potent mitogens for a variety of cells of mesodermal, ectodermal, and endodermal origin, including fibroblasts, corneal and vascular endothelial cells, granulocytes, adrenal cortical cells, chondrocytes, myoblasts, vascular smooth muscle cells, lens epithelial cells, melanocytes, keratinocytes, oligodendrocytes, astrocytes, osteoblasts, and hematopoietic cells.

FGF-1 and FGF-2 were isolated from brain and pituitary as mitogens to stimulate fibroblast proliferation (Burgess et al., supra). In addition to the ability to stimulate proliferation of vascular endothelial cells, both FGF-1 and 2 are chemotactic for endothelial cells and FGF-2 has been shown to enable endothelial cells to penetrate the basement membrane. Consistent with these properties, both FGF-1 and 2 have the capacity to stimulate angiogenesis. Another important feature of these growth factors is their ability to promote wound healing.

FGF-3, 4, 5, 6 were isolated as oncogenes (Dickson, C., et al., Ann. NY Acad. Sci. 638: 18-26 (1991); Yoshida, T., et al., Ann. NY Acad. Sci. 638: 27-37 (1991); Goldfarb, M., et al., Ann. NY Acad. Sci. 638: 38-52 (1991); Coulier, F., et al., Ann. NY Acad. Sci. 638: 53-61 (1991). FGF-3 is pivotal to mouse inner ear development (Mansour, S. L., et al., Development 117: 13-28 (1993)). FGF-4 was isolated from a human stomach tumor as hst-1, and from a Kaposi's sarcoma as k-FGF (Delli-Bovi, P., et al., Mol. Cell Biol. 8: 2933-2941 (1988)). In the human, int-2 and FGF-4 (k-FGF) are situated in close proximity on the short arm of chromosome 11 and in certain types of tumors both genes are amplified in concert.

FGF-5, a human oncogene, was isolated from a human bladder tumor ( Zhan, X., et al., Mol. Cell Biol. 8: 3487-3495 (1988)). FGF-5 has been shown to inhibit hair growth (Hebert, J. M., et al., Cell 78, 1017-1025 (1994)). FGF-6 was identified by systemically screening a mouse cosmid library with a human hst probe (Quarto, N., et al, Oncogene Res. 5:101-110 (1989)).

FGF-7 was isolated as a proliferating factor for isolated keratinocytes (Aaronson, S. A., et al., Ann. NY Acad. Sci. 638: 62-77 (1991). FGF-7 controls hair follicle morphogenesis and hair structure (Guo, L., et al., EMBO J. 12, 973-986 (1993)). Another utility of KGF (FGF-7) as a type II alveolar epithelial cell differentiation factor was well documented by Ulich, T. R., et al., J. Clin. Invest. 93:1298-1306 (1994); and by Guo, J., et al., Am J. Physiol. 275 (Lung Cell Mol. Physiol. 19): L800-L805 (1998), who found that intravenous administration of KGF stimulates proliferation of alveolar and bronchial epithelial cells, and protected against experimental lung injury.

FGF-8 was isolated as androgen-induced growth factor (AIGF) (Tanaka, A., et al., Proc. Natl. Acad. Sci. USA 89: 8928-8932 (1992)). AIGF is a distinctive FGF-like growth factor, having a putative signal peptide and sharing 30-40% homology with known members of the FGF family. Mammalian cells transformed with AIGF show a remarkable stimulatory effect on the growth of SC-3 cells in the absence of androgen. Therefore, AIGF mediates androgen-induced growth of SC-3 cells, and perhaps other cells, since it is secreted by the tumor cells themselves.

FGF-9 is sometimes referred to as Glial Activating Factor (GAF) because it was purified from the culture supernatant from a human glioma cell line. (Miyamoto, M. et al., Mol. and Cell. Biol., 13(7):4251-4259 (1993)). FGF-9 has around 30% sequence similarity to other members of the FGF family. Two cysteine residues and other consensus sequences in family members were also well conserved in the FGF-9 sequence. FGF-9 was found to have no typical signal sequence in its N terminus like those in acidic and basic FGF. However, FGF-9 was found to be secreted from cells after synthesis despite its lack of a typical signal sequence. Further, FGF-9 was found to stimulate the cell growth of oligodendrocyte type 2 astrocyte progenitor cells, BALB/c3T3, and PC-12 cells but not that of human umbilical vein endothelial cells (Naruo, K., et al., J. Biol.Chem., 268:2857-2864 (1993).

FGF-10, 11, 12, 13, 14, 16 and 17 were discovered by database search and homology-based PCR (Yamasaki, M., et al., J. Biol. Chem. 271: 15918-15921 (1996); Smallwood, P. M., et al., Proc. Natl. Acad. Sci. USA 93: 9850-9857 (1996); Miyake, A., et al., Biochem. Biophys. Res. Commun. 243(1): 148-152 (1998); Hoshikawa, M., et al., Biochem. Biophys. Res. Commun. 244: 187-191 (1998). FGF-15 was identified by representational difference analysis as a result of its inductive expression by the chimeric homeodomain oncoprotein E2A-Pbx1 (McWhirter, J. R., et al. Development 124: 3221-3232 (1997).

FGF-18 was identified using a novel EST with homology to human FGFs-8 and 9 as a probe to screen a mouse kidney cDNA library (Hu, M. C-T., et al., Mol. Cell Biol. 18(10):6063-6074 (1998). Mouse and human FGF-18 stimulated growth in a number of tissues in mouse, most notably in the liver and small intestine (Hu, M. C.-T., et al., Mol. Cell Biol. 18(10): 6063-6074 (1998).

Another fibroblast growth factor, FGF-19, has recently been identified. (See International Patent Publications WO 99/14327 and WO 99/27100). This fibroblast growth factor, also termed PRO533, was identified as a gene that is amplified in the genome of certain tumor cells. In other words, this growth factor was found to contribute to tumorigeneis. (See International Patent Publication WO 99/27100).

Each member of the fibroblast growth factor family has functions that overlap with the others, in addition to having their own unique spectrum of functions. Further characterization of these growth factors is, therefore an important step in understanding their unique and diverse roles, particularly as they relate to angiogenesis.

Persistent, unregulated angiogenesis occurs in a multiplicity of disease states, including tumor metastasis and abnormal growth by endothelial cells, and supports the pathological damage seen in these conditions. Thus, characterization of angiogenic factors may also facilitate the development of treatments for diseases related to (and hypothesized as being related to) angiogenesis. For example, tumor formation has been proposed to be dependent on angiogenesis. Thus, fibroblast growth factors that inhibit angiogenesis may provide therapeutic treatments for such tumors.

### SUMMARY OF THE INVENTION

The present invention provides methods for modulating angiogenesis using anti-angiogenic FGF-19 polypeptides. The present invention further encompasses the use of FGF-19 polypeptides for the treatment of a disease or clinical condition where angiogenesis is relevant to the causation or treatment of the disease or clinical condition. In one embodiment, such diseases or conditions include, but are not limited to, cancer, wound healing, tumor formation, diabetic retinopathies, macular degeneration, cardiovascular diseases, and the like. Further uses of the FGF-19 polypeptides include treatment of clinical conditions involving angiogenesis in the reproductive system, including regulation of placental vascularization or use as an abortifacient. The present invention also encompasses pharmaceutical compositions containing the FGF-19 polypeptide and the use of such pharmaceutical compositions for the treatment of the above-mentioned diseases or clinical conditions.

One aspect of the present invention relates to the use of FGF-19 polypeptides having the amino acid sequence of SEQ ID NO:2, as well as biologically active or diagnostically or therapeutically useful fragments, variants, derivatives and analogs thereof. In a related aspect, the FGF-19 polypeptides of the present invention are identified in International Patent Publication WO 99/27100, the disclosure of which is incorporated herein in its entirety. An additional aspect relates to the use of antibodies against the FGF-19 polypeptides of the present invention, especially antibodies which bind specifically to an epitope of the sequence described in SEQ ID NO:2, or a sequence that shares at least 60%, preferably at least 70%, more preferably at least 80%, still more preferably at least 90%, or most preferably at least 95% sequence identity over at least 20, preferably at least 30, more preferably at least 40, still more preferably at least 50, or most preferably at least 100 residues, to SEQ ID NO:2.

Another aspect of the present invention relates to the use of isolated FGF-19 nucleic acids encoding the FGF-19 polypeptides of the present invention, including mRNAs, DNAs, cDNAs, genomic DNA, as well as FGF-19 antisense nucleic acids. Such nucleic acids include the FGF-19 cDNA sequence having the nucleotide sequence of SEQ ID NO:1. Another aspect relates to FGF-19 sequence fragments or variants that encode biologically active or diagnostically or therapeutically useful polypeptides. Such fragments or variants include sequences having all possible codon choices for the same amino acid or conservative amino acid substitutions thereof. Other variants include those nucleic acids that are capable of selectively hybridizing to a human FGF-19 cDNA (e.g., SEQ ID NO:1) under stringent hybridization conditions. Another aspect of the present invention relates to nucleic acid probes comprising polynucleotides of sufficient length to selectively hybridize to a polynucleotide encoding an FGF-19 polypeptide of the present invention.

Still another aspect of the present invention relates to processes for producing FGF-19 polypeptides, or biologically active and diagnostically or therapeutically useful fragments or variants thereof, by recombinant techniques through the use of recombinant vectors. A further aspect of the present invention relates to recombinant prokaryotic and/or eukaryotic host cells comprising an FGF-19 nucleic acid sequence encoding an FGF-19 polypeptide, or biologically active or diagnostically or therapeutically useful fragments or variants thereof. In a related aspect, nucleic acid constructs are provided that express FGF-19 nucleic acids and/or FGF-19 polypeptides, fragments or variants. Such constructs typically include a transcriptional promoter and a transcriptional terminator, each operably linked for expression of the FGF-19 nucleic acid or fragment thereof.

Another aspect of the present invention relates to processes involving expression of the polypeptides, or polynucleotides encoding the polypeptides, of the present invention for purposes of gene therapy. As used herein, gene therapy is defined as the process of providing for the expression of nucleic acid sequences of exogenous origin in an individual for the treatment of a disease condition within that individual.

A further aspect of the present invention relates to processes for utilizing FGF-19 polypeptides fragments, variants, derivatives, or analogs thereof, or FGF-19 polynucleotides or fragments, variants or derivatives thereof, for therapeutic purposes involving the modulation of angiogenesis, or the modulation of diseases or conditions in which angiogenesis is relevant to the disease or condition. Such diseases or conditions include, for example, the treatment of cancer, wound healing, diabetic retinopathies, macular degeneration, cardiovascular diseases, and clinical conditions involving angiogenesis in the reproductive system, including regulation of placental vascularization or use as an abortifacient. Such treatments further include the use of the FGF-19 polypeptides in protein replacement therapy and protein mimetics.

Another aspect of the present invention relates to diagnostic assays for detecting diseases or clinical conditions, or the susceptibility to diseases or clinical conditions, related to mutations in an FGF-19 nucleic acid sequence of the present invention and for detecting over-expression or underexpression of FGF-19 polypeptides encoded by such sequences.

These and other aspects of the invention will become evident upon reference to the following description and drawings.

### BRIEF DESCRIPTION OF THE DRAWINGS

FIG. 1 depicts the cDNA sequence (SEQ ID NO:1) and corresponding deduced amino acid sequence (SEQ ID NO:2) of FGF-19.

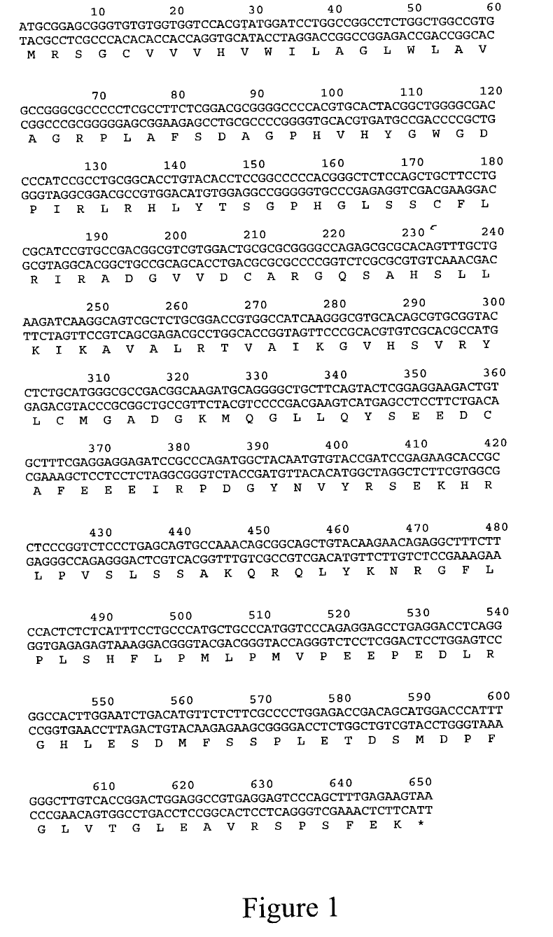

FIG. 2 is an autoradiogram of a gel from a Western Blot assay showing the transient expression of human FGF-19 in HEK-293 cells.

### DETAILED DESCRIPTION

Prior to setting forth the invention in more detail, it may be helpful to a further understanding thereof to set forth definitions of certain terms as used hereinafter.

Definitions:

Unless defined otherwise, all technical and scientific terms used herein have the same meaning as commonly understood by one of ordinary skill in the art to which this invention belongs. Other methods and materials similar to those described herein can be used in the practice or testing of the present invention; thus, only exemplary methods and materials are described. For purposes of the present invention, the following terms are defined below.

The term “angiogenesis” means the generation of new blood vessels in a tissue or organ. Angiogenesis includes neovascularization and collateral vascularization. “Normal angiogenesis” includes, under normal physiological conditions, new blood vessel formation associated with wound healing, fetal and embryonal development and formation of the corpus luteum, endometrium and placenta. “Unwanted angiogenesis” refers to angiogenesis that occurs under abnormal physiological conditions, such as in a disease or clinical condition associated with pathological damage related to the uncontrolled angiogenesis. For example, unwanted angiogenesis can occur during tumor formation, where neovascularization is present within the tumor.

The term “FGF-19 nucleic acids” (i.e., in all caps and italicized) refers to polynucleotides encoding FGF-19 polypeptides, including mRNAs, DNAs, cDNAs, genomic DNA, as well as antisense nucleic acids, and polynucleotides encoding biologically active or diagnostically or therapeutically useful fragments, variants and derivatives thereof. Useful fragments and variants include those based on all possible codon choices for the same amino acid, and codon choices based on conservative amino acid substitutions, and biologically active or diagnostically or therapeutically useful fragments, or derivatives thereof. Useful variants further include those having at least 70% polynucleotide sequence identity, more preferably 80%, still preferably 90%, to the polynucleotide of SEQ ID NO:1, and biologically active and diagnostically or therapeutically useful fragments, or derivatives thereof.

The term “FGF-19 gene” (i.e., in all caps and italicized) refers to coding sequences, intervening sequences and regulatory elements controlling transcription and/or translation.

The terms “polynucleotide” and “nucleic acid” refer to a polymer composed of a multiplicity of nucleotide units (ribonucleotide or deoxyribonucleotide or related structural variants), which are typically linked via phosphodiester bonds. A polynucleotide or nucleic acid can be of substantially any length, typically from about six (6) nucleotides to about 10⁹ nucleotides or larger. Polynucleotides or nucleic acids include RNA, cDNA, genomic DNA, synthetic forms, and mixed polymers, both sense and antisense strands, and can also be chemically or biochemically modified or can contain non-natural or derivatized nucleotide bases, as will be readily appreciated by the skilled artisan. Such modifications include, for example, labels, methylation, substitution of one or more of the naturally occurring nucleotides with an analog, intemucleotide modifications such as uncharged linkages (e.g., methyl phosphonates, phosphotriesters, phosphoamidates, carbamates, and the like), charged linkages (e.g., phosphorothioates, phosphorodithioates, and the like), pendent moieties (e.g, polypeptides), intercalators (e.g., acridine, psoralen, and the like), chelators, alkylators, and modified linkages (e.g., alpha anomeric nucleic acids, and the like). Also included are synthetic molecules that mimic polynucleotides in their ability to bind to a designated nucleotide sequence via hydrogen bonding and other chemical interactions. Such molecules are known in the art and include, for example, those in which peptide linkages substitute for phosphate linkages in the backbone of the molecule.

The term “oligonucleotide” refers to a polynucleotide of from about six (6) to about one hundred (100) nucleotides, or more in length. Thus, oligonucleotides are a subset of polynucleotides. Oligonucleotides can be synthesized on an automated oligonucleotide synthesizer (e.g., those manufactured by Applied BioSystems (Foster City, Calif.)), according to specifications provided by the manufacturer.

The term “primer” refers to a polynucleotide, typically an oligonucleotide, whether occurring naturally, as in an enzyme digest, or produced synthetically in vitro, which acts as a point of initiation of polynucleotide synthesis when used under conditions in which a primer extension product is synthesized.

The term “FGF-19 polypeptide” refers to polypeptides having the amino acid sequence of SEQ ID NO:2, and biologically active or diagnostically or therapeutically useful fragments, variants and derivatives thereof. “Fragment” refers to a portion of an FGF-19 polypeptide having typically at least 10 contiguous amino acids, more typically at least 20, still more typically at least 50 contiguous amino acids of the FGF-19 polypeptide. Useful variants typically include those having conservative amino acid substitutions, and biologically active and diagnostically or therapeutically useful fragments thereof. Useful variants are typically at least about 50% similar to the native FGF-19 amino acid sequence (SEQ ID NO:2), more typically in excess of about 90%, and still more typically at least about 95% similar, and biologically active, or diagnostically or therapeutically useful fragments or derivatives thereof. FGF-19 polypeptides further include those that are immunologically cross-reactive with anti-FGF-19 polypeptides. FGF-19 polypeptides still further include fusion proteins.

The term “polypeptide” refers to a polymer of amino acids and its equivalent and does not refer to a specific length of the product; thus, peptides and oligopeptides (i.e., fragments) and proteins are included within the definition of a polypeptide. This term also includes derivatives of the FGF-19 polypeptide, for example, glycosylations, acetylations, phosphorylations, and the like. Included within the definition are, for example, polypeptides containing one or more analogs of an amino acid (e.g, unnatural amino acids, and the like), polypeptides with substituted linkages as well as other modifications known in the art, both naturally and non-naturally occurring.

The term “biologically active” refers to the ability of a molecule to modulate angiogenesis, such as by affecting endothelial tube formation (e.g., using the HUVEC assays described in Examples 3 & 4 (infra)), or that affects tumor cell growth or proliferation (e.g., using the tumor cell growth inhibition assays of Examples 5 and 6 (infra)). Biologically active molecules can be FGF-19 polypeptides, fragments, variants, derivatives and analogs thereof; nucleic acids encoding FGF-19 polypeptides, fragments, variants and derivatives thereof; and anti-FGF-19 antibodies, which modulate angiogenesis (e.g., inhibiting or stimulating endothelial tube formation) or by modulating tumor cell growth or proliferation (e.g., inhibiting or stimulating tumor cell growth).

The terms “therapeutically useful” or “therapeutically effective” refer to an amount of a molecule (e.g., an FGF-19 polypeptide, anti-FGF-19 antibody, or FGF-19 nucleic acid) that is sufficient to modulate angiogenesis (e.g., inhibiting or stimulating endothelial tube formation) or to modulate tumor cell growth or proliferation (e.g., inhibiting or stimulating tumor cell growth) in a subject, such as a patient or a mammal.

The terms “diagnostically useful” or “diagnostically effective” refer to a molecule (e.g., an FGF-19 polypeptide, anti-FGF-19 antibody, or FGF-19 nucleic acid) for detecting angiogenesis, or the inhibition of angiogenesis, in a subject. These terms further include molecules useful for detecting diseases or clinical conditions, or the susceptibility to diseases or clinical conditions, related to mutations in an FGF-19 nucleic acid sequence of the present invention and for detecting over-expression or underexpression of FGF-19 polypeptides encoded by such sequences.

The term “FGF-19 compounds” or “FGF-19 anti-angiogenic compounds” refers to biologically active FGF-19 polypeptide, fragments, variants, derivatives, or analogs thereof, to anti-FGF-19 antibodies, to biologically active FGF-19 nucleic acids, fragments or derivatives, and to FGF-19 antisense nucleic acids.

The terms “amino acid,” “amino acid residue,” or “residue” refer to naturally occurring L amino acids or to D amino acids as described further below. Amino acids are referred to herein by either their commonly known three letter symbols or by the one-letter symbols recommended by the IUPAC-IUB Biochemical Nomenclature Commission. Nucleotides, likewise, may be referred to by their commonly accepted single-letter codes. (See, e.g., Bruce Alberts et al., Molecular Biology of the Cell, Garland Publishing, Inc., New York (3d ed. 1994)).

The terms “identical” or “percent identity,” in the context of two or more nucleic acids or polypeptide sequences, refer to two or more sequences or subsequences that are the same or have a specified percentage of nucleotides or amino acids that are the same, when compared and aligned for maximum correspondence, as measured using one of the following sequence comparison algorithms, or by visual inspection.

The term “substantially identical,” in the context of two nucleic acids, or two polypeptide sequences, refers to two or more sequences or subsequences that have at least 60%, typically 80%, most typically 90-95% identity, when compared and aligned for maximum correspondence, as measured using one of the sequence comparison algorithms described below, or by visual inspection. An indication that two polypeptide sequences are “substantially identical” is that one polypeptide is immunologically reactive with antibodies raised against the second polypeptide.

“Similarity” or “percent similarity” in the context of two or polypeptide sequences, refer to two or more sequences or subsequences that are the same or have a specified percentage of amino acid residues or conservative substitutions thereof, that are the same, when compared and aligned for maximum correspondence, as measured using one of the following sequence comparison algorithms, or by visual inspection. By way of example, a first protein region can be considered similar to a region of human FGF-19 polypeptide when the amino acid sequence of the first region is at least 30%, 40%, 50%, 60%, 70%, 75%, 80%, 90%, or even 95% identical, or conservatively substituted, to a region of FGF-19 polypeptide when compared to any sequence in FGF-19 polypeptide of an equal number of amino acids as the number contained in the first region, or when compared to an aligned sequence of FGF-19 polypeptide that has been aligned by a computer similarity program known in the art, as discussed above.

The term “substantial similarity” in the context of polypeptide sequences, indicates that the polypeptide comprises a sequence with at least 70% sequence identity to a reference sequence, or preferably 80%, or more preferably 85% sequence identity to the reference sequence, or most preferably 90% identity over a comparison window of about 10-20 amino acid residues. In the context of amino acid sequences, “substantial similarity” further includes conservative substitutions of amino acids. Thus, a polypeptide is substantially similar to a second polypeptide, for example, where the two peptides differ only by one or more conservative substitutions.

The term “conservative substitution,” when describing a polypeptide, refers to a change in the amino acid composition of the polypeptide that does not substantially alter the polypeptide's activity. Thus, a “conservative substitution” of a particular amino acid sequence refers to amino acid substitutions of those amino acids that are not critical for protein activity or substitution of amino acids with other amino acids having similar properties (e.g., acidic, basic, positively or negatively charged, polar or non-polar, etc.) such that the substitutions of even critical amino acids do not substantially alter activity. Conservative substitution tables providing functionally similar amino acids are well known in the art. The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

(See also Creighton, Proteins, W. H. Freeman and Company (1984).) In addition, individual substitutions, deletions or additions that alter, add or delete a single amino acid or a small percentage of amino acids in an encoded sequence are also “conservative substitutions.”

For sequence comparison, typically one sequence acts as a reference sequence, to which test sequences are compared. When using a sequence comparison algorithm, test and reference sequences are input into a computer, coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm of Smith & Waterman (Adv. Appl. Math. 2:482 (1981)), by the homology alignment algorithm of Needleman & Wunsch (J. Mol. Biol. 48:443 (1970)), by the search for similarity method of Pearson & Lipman (Proc. Natl. Acad. Sci. USA 85:2444 (1988)), by computerized implementations of these algorithms (e.g., GAP, BESTFIT, FASTA, and TFASTA in the Wisconsin Genetics Software Package, Genetics Computer Group, 575 Science Dr., Madison, Wis.), or by visual inspection. (See generally Ausubel et al., Current Protocols in Molecular Biology, John Wiley and Sons, New York (1996).)

One example of a useful algorithm is PILEUP. PILEUP creates a multiple sequence alignment from a group of related sequences using progressive, pairwise alignments to show the percent sequence identity. It also plots a tree or dendogram showing the clustering relationships used to create the alignment. PILEUP uses a simplification of the progressive alignment method of Feng & Doolittle (J. Mol. Evol. 35:351-360 (1987)). The method used is similar to the method described by Higgins & Sharp (CABIOS 5:151-153 (1989)). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps. Another useful program for the multiple alignment of sequences is MEGALIGN™ Expert Sequence Analysis Software (DNASTAR, Madison, Wis.).

Another example of an algorithm that is suitable for determining percent sequence identity and similarity is the BLAST algorithm, which is described by Altschul et al. (J. Mol. Biol. 215:403-410 (1990)). (See also Zhang et al., Nucleic Acid Res. 26:3986-90 (1998); Altschul et al., Nucleic Acid Res. 25:3389-402 (1997). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990), supra). These initial neighborhood word hits act as seeds for initiating searches to find longer HSPs containing them. The word hits are then extended in both directions along each sequence for as far as the cumulative alignment score can be increased. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff & Henikoff, Proc. Natl. Acad. Sci. USA 89:10915-9 (1992)), alignments (B) of 50, expectation (E) of 10, M=5, N=−4, and a comparison of both strands.

In addition to calculating percent sequence identity, the BLAST algorithm also performs a statistical analysis of the similarity between two sequences (see, e.g., Karlin & Altschul, Proc. Natl. Acad. Sci. USA 90:5873-77 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more typically less than about 0.01, and most typically less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two polypeptides differ only by conservative substitutions.

The terms “transformation” or “transfection” means the process of stably altering the genotype of a recipient cell or microorganism by the introduction of polynucleotides. This is typically detected by a change in the phenotype of the recipient cell or organism. The term “transformation” is generally applied to microorganisms, while “transfection” is used to describe this process in cells derived from multicellular organisms.

Methodologies for polymerase chain reaction (“PCR”) are generally disclosed in U.S. Pat. Nos. 4,683,202, 4,683,195 and 4,800,159. Other nomenclature used herein and many of the laboratory procedures in cell culture, molecular genetics and nucleic acid chemistry and hybridization, which are described below, are those well known and commonly employed in the art. (See generally Ausubel et al., Current Protocols in Molecular Biology, John Wiley and Sons, New York (1996); Sambrook et al., Molecular Cloning: A Laboratory Manual, Second Edition, Cold Spring Harbor Laboratory Press, New York (1989)). Standard techniques are used for recombinant nucleic acid methods, polynucleotide synthesis, preparation of biological samples, preparation of cDNA fragments, isolation of mRNA and the like. Generally enzymatic reactions and purification steps are performed according to the manufacturers' specifications.

FGF-19 Nucleic Acids:

One aspect of the present invention relates to isolated nucleic acids encoding FGF-19 polypeptides, including mRNAs, DNAs, cDNAs, genomic DNA, as well as antisense nucleic acids, and their use in modulating angiogenesis. FGF-19 nucleic acids include the FGF-19 cDNA sequence (e.g., SEQ ID NO:1). FGF-19 nucleic acids further include biologically active sequence variants, such as those encoding all possible codon choices for the same amino acid or conservative amino acid substitutions thereof, and also include diagnostically or therapeutically useful fragments thereof.

The invention further provides purified FGF-19 nucleic acids comprising at least 6 contiguous nucleotides (e.g., a hybridizable portion) encoding a fragment of an FGF-19 polypeptide. In another embodiment, the FGF-19 nucleic acids consist of fragments of at least 8 (contiguous) nucleotides, 25 nucleotides, 50 nucleotides, 100 nucleotides, 150 nucleotides, 200 nucleotides, or even up to 250 nucleotides or more of an FGF-19 sequence. In another embodiment, the nucleic acids are larger than 200 or 250 nucleotides in length. The nucleic acids can be single- or double-stranded. As is readily apparent, as used herein, a “nucleic acid encoding a fragment of an FGF-19 polypeptide” is construed as referring to a nucleic acid encoding only the recited fragment or portion of the FGF-19 polypeptide and not the other contiguous portions of the FGF-19 polypeptide as a continuous sequence. Fragments of FGF-19 nucleic acids encoding one or more FGF-19 domains are provided.

The invention also relates to nucleic acids hybridizable to, or complementary to, the foregoing sequences. Such nucleic acids include mRNAs, DNAs, cDNAs, genomic DNA, as well as antisense nucleic acids, and biologically active and diagnostically or therapeutically useful fragments or variants thereof. Nucleic acids are also provided which comprise a sequence complementary to at least 10, 25, 50, 100, 200, or 250 nucleotides or more of an FGF-19 gene or cDNA. In one embodiment, a nucleic acid is hybridizable to an FGF-19 nucleic acid (e.g., having sequence SEQ ID NO:1), or to a nucleic acid encoding an FGF-19 variant, under conditions of high stringency is provided.

By way of example, and not limitation, procedures using conditions of high stringency are as follows: Prehybridization of filters containing DNA is carried out for 8 hours to overnight at 65° C. in buffer composed of 6×SSC, 50 mM Tris-HCl (pH 7.5), 1 mM EDTA, 0.02% PVP, 0.02% Ficoll, 0.02% BSA, and 500 (μg/ml denatured salmon sperm DNA). Filters are hybridized for 48 hours at 65° C. in prehybridization mixture containing 100 μg/ml denatured salmon sperm DNA and 5-20×10⁶ cpm of ³²P-labeled probe. Washing of filters is done at 37° C. for 1 hour in a solution containing 2×SSC, 0.01% PVP, 0.01% Ficoll, and 0.01% BSA. This is followed by a wash in 0.1×SSC at 50° C. for 45 min before autoradiography. Other conditions of high stringency, which can be used, are well known in the art. (See generally Ausubel et al., supra).

In another embodiment, a nucleic acid which is hybridizable to an FGF-19 nucleic acid under conditions of moderate stringency is provided. By way of example, and not limitation, procedures using such conditions of moderate stringency are as follows: Prehybridization of filters containing DNA is carried out for 8 hours to overnight at 55° C. in buffer composed of 6×SSC, 50 mM Tris-HCl (pH 7.5), 1 mM EDTA, 0.02% PVP, 0.2% Ficoll, 0.02% BSA and 500 μg/ml denatured salmon sperm DNA. Filters are hybridized for 24 hours at 55° C. in a prehybridization mixture containing 100 μg/ml denatured salmon sperm DNA and 5-20×10⁶ cpm of ³²P-labeled probe. Washing of filters is done at 37° C. for 1 hour in a solution containing 2×SSC, 0.01% PVP, 0.01% Ficoll, and 0.01% BSA.

In another embodiment, a nucleic acid which is hybridizable to an FGF-19 nucleic acid under conditions of low stringency is provided. By way of example, and not limitation, procedures using such conditions of low stringency are as follows: Filters containing DNA are pretreated for 6 hours at 40° C. in a solution containing 35% formamide, 5×SSC, 50 mM Tris-HCl (pH 7.5), 5 mM EDTA, 0.1% polyvinylpyrrolidone (PVP), 0.1% Ficoll, 1% bovine serum albumin (BSA), and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe. Filters are incubated in hybridization mixture for 18-20 hours at 40° C., and then washed for 1.5 hours at 55° C. in a solution containing 2×SSC, 25 mM Tris-HCl (pH 7.4), 5 mM EDTA, and 0.1% SDS. The wash solution is replaced with fresh solution and incubated an additional 1.5 hours. Filters are blotted dry and exposed for autoradiography. Other conditions of low stringency that can be used are well known in the art (e.g., those employed for cross-species hybridizations). (See also Shilo and Weinberg, Proc. Natl. Acad. Sci. USA 78:6789-6792 (1981)).

Low, moderate and high stringency conditions are well known to those of skill in the art, and will vary predictably depending on the base composition of the particular nucleic acid sequence and on the specific organism from which the nucleic acid sequence is derived. For guidance regarding such conditions see, for example, Sambrook et al., Molecular Cloning, A Laboratory Manual (Second Edition, Cold Spring Harbor Press, NY, pp. 9.47-9.57 (1989)); and Ausubel et al., Current Protocols in Molecular Biology (Green Publishing Associates and Wiley Interscience, NY (1989)).

Specific embodiments for the cloning of an FGF-19 nucleic acid, presented as a particular example but not by way of limitation, are as follows.

For expression cloning (a technique commonly known in the art), an expression library is constructed by methods known in the art. For example, mRNA (e.g., human) is isolated, cDNA is prepared and then ligated into an expression vector (e.g., a bacteriophage derivative) such that it is capable of being expressed by the host cell into which it is then introduced. Various screening assays can then be used to select for the expressed FGF-19 polypeptide. In one embodiment, anti-FGF-19 specific antibodies can be used for selection.

In another embodiment, polymerase chain reaction (PCR) is used to amplify the desired sequence in a genomic or cDNA library prior to selection. Oligonucleotides representing known FGF-19 sequences, for example, as selected from SEQ ID NO:1, can be used as primers in PCR. In a typical embodiment, the oligonucleotide represents at least part of the FGF-19 conserved segments of sequence identity between FGF-19 of different species. The synthetic oligonucleotides can be utilized as primers to amplify particular sequences within an FGF-19 gene by PCR using nucleic acids from a source (RNA or DNA), typically a cDNA library, of potential interest. PCR can be carried out, for example, by use of a Perkin-Elmer Cetus thermal cycler and Taq polymerase (Gene Amp). The DNA being amplified can include mRNA or cDNA or genomic DNA from any eukaryotic species. One of skill in the art can choose to synthesize several different degenerate primers for use in the PCR reactions.

It is also possible to vary the stringency of hybridization conditions used in priming the PCR reactions, to allow for greater or lesser degrees of nucleotide sequence similarity between the known FGF-19 sequence and the related nucleic acid being isolated. For cross species hybridization, low stringency conditions are typically used. For same species hybridization, moderately stringent conditions are more typically used. After successful amplification of a segment of a related FGF-19 nucleic acid, that segment can be molecularly cloned and sequenced, and utilized as a probe to isolate a complete cDNA or genomic clone. This, in turn, can permit the determination of a complete nucleotide sequence, the analysis of its expression, and the production of its protein product for functional analysis, as described infra. In this fashion, additional nucleic acids encoding FGF-19 polypeptides and FGF-19 polypeptide variants can be identified.

The above-methods are not meant to limit the following general description of methods by which FGF-19 nucleic acids can be obtained. Any eukaryotic cell potentially can serve as the nucleic acid source for the molecular cloning of FGF-19 nucleic acids. The nucleic acids encoding FGF-19 polypeptide can be isolated from vertebrate sources including, mammalian sources such as, porcine, bovine, feline, avian, equine, canine and human as well as additional primate sources. The DNA can be obtained by standard procedures known in the art from cloned DNA (e.g., a DNA “library”), by chemical synthesis, by cDNA cloning, or by the cloning of genomic DNA, or fragments thereof, purified from the desired cell. (See, e.g., Sambrook et al., Molecular Cloning, A Laboratory Manual, Second Edition, Cold Spring Harbor Laboratory Press, Cold Spring Harbor, N.Y. (1989); Glover, (ed.), DNA Cloning: A Practical Approach, MRL Press, Ltd., Oxford, U.K. Vol. I, II. (1985)). Clones derived from genomic DNA can contain regulatory and intron DNA regions in addition to coding regions; clones derived from cDNA will typically contain only exon sequences. Whatever the source, the nucleic acid can be molecularly cloned into a suitable vector for propagation of the nucleic acid.

In the molecular cloning of FGF-19 nucleic acids from genomic DNA, DNA fragments are generated, some of which will encode an FGF-19 gene. The DNA can be cleaved at specific sites using various restriction enzymes. Alternatively, one can use DNase in the presence of manganese to fragment the DNA, or the DNA can be physically sheared, such as, for example, by sonication. The linear DNA fragments can then be separated according to size by standard techniques, including but not limited to, agarose and polyacrylamide gel electrophoresis and column chromatography.

Once the DNA fragments are generated, identification of the specific DNA fragment containing the desired gene can be accomplished in a number of ways. For example, a portion of an FGF-19 gene, cDNA (of any species) or its specific RNA, or a fragment thereof, can be purified and labeled, the generated DNA fragments can be screened by nucleic acid hybridization to the labeled probe (see, e.g., Benton and Davis, Science 196:180 (1975); Grunstein and Hogness, Proc. Natl. Acad. Sci. USA 72:3961 (1975)). Those DNA fragments with substantial identity to the probe will hybridize. It is also possible to identify the appropriate fragment by restriction enzyme digestion(s) and comparison of fragment sizes with those expected according to a known restriction map, if available. Further selection can be carried out on the basis of the properties of the gene.

Alternatively, the presence of the gene can be detected by assays based on the physical, chemical, or immunological properties of its expressed product. For example, cDNA clones, or DNA clones which hybrid-select the proper mRNAs, can be selected which produce a polypeptide that, for example, has similar or identical electrophoretic migration, isoelectric focusing behavior, proteolytic digestion maps, modulation of angiogenesis, receptor binding activity, or antigenic properties as known for FGF-19 polypeptide. Immune serum or an antibody which specifically binds to the FGF-19 polypeptide can be used to identify putatively FGF-19 polypeptide synthesizing clones by binding in an ELISA (enzyme-linked immunosorbent assay)-type procedure.

FGF-19 nucleic acids can also be identified by mRNA selection by nucleic acid hybridization followed by in vitro translation. In this procedure, fragments are used to isolate complementary mRNAs by hybridization. Such DNA fragments typically represent available, purified DNA of another species (e.g., human, mouse, and the like). Immunoprecipitation analyses or functional assays (e.g., inhibition of angiogenesis, endothelial tube formation in vitro or tumor inhibition) of the in vitro translation products of the isolated mRNAs identifies the mRNA and, therefore, the complementary DNA fragments that contain the desired sequences. In addition, specific mRNAs can be selected by adsorption of polysomes isolated from cells to immobilized antibodies specifically directed against FGF-19 polypeptide. A radiolabeled FGF-19 cDNA can be synthesized using the selected mRNA (from the adsorbed polysomes) as a template. The radiolabeled mRNA or cDNA can then be used as a probe to identify the FGF-19 nucleic acid fragments from among other genomic DNA fragments.

Alternatives to isolating the FGF-19 genomic DNA include, but are not limited to, chemically synthesizing the gene sequence itself from a known sequence or making cDNA to mRNA that encodes an FGF-19 polypeptide. For example, RNA for cDNA cloning of the FGF-19 gene can be isolated from cells that express the FGF-19 polypeptide. Other methods are possible.

The identified and isolated FGF-19 nucleic acids can then be inserted into an appropriate cloning vector. A large number of vector-host systems known in the art can be used. Possible vectors include, but are not limited to, plasmids or modified viruses. The vector system is selected to be compatible with the host cell. Such vectors include, but are not limited to, bacteriophages such as lambda derivatives, yeast integrative and centromeric vectors, 2μ plasmid, and derivatives thereof, or plasmids such as pBR322, pUC, pcDNA3 or pRSETC (InVitrogen, San Diego, Calif.) plasmid derivatives or the Bluescript vector (Stratagene, La Jolla, Calif.), to name a few. The insertion of the FGF-19 nucleic acids into a cloning vector can be accomplished, for example, by ligating the DNA fragment into a cloning vector which has complementary cohesive termini. If the complementary restriction sites used to fragment the DNA are not present in the cloning vector, however, the ends of the DNA molecules can be enzymatically modified. Alternatively, any site desired can be produced by ligating nucleotide sequences (linkers) onto the DNA termini; these ligated linkers can comprise specific chemically synthesized oligonucleotides encoding restriction endonuclease recognition sequences. A restriction site can also be introduced into a nucleic acid by PCR amplification of the nucleic acid using a primer(s) that encodes the desired restriction site(s). In an alternative method, the cleaved vector and FGF-19 nucleic acids can be modified by homopolymeric tailing. Recombinant molecules can be introduced into host cells via transformation, transfection, infection, electroporation, and the like, so that many copies of the gene sequence are generated.

In another method, the FGF-19 gene can be identified and isolated after insertion into a suitable cloning vector in a “shot gun” approach. Enrichment for the FGF-19 gene, for example, by size fractionation, can be done before insertion into the cloning vector. In specific embodiments, transformation of host cells with recombinant DNA molecules that incorporate the isolated FGF-19 gene, cDNA, or synthesized DNA sequence enables generation of multiple copies of the gene. Thus, the gene can be obtained in large quantities by growing transformants, isolating the recombinant DNA molecules from the transformants and, when necessary, retrieving the inserted gene from the isolated recombinant DNA.

A clone harboring the FGF-19 cDNA, pcDNA3-FGF-19-HA, was deposited at the American Type Culture Collection (“ATCC”), 12301 Parklawn Drive, Rockville, Md. 20852 U.S.A., on Dec. 21, 1998, under Deposit Registration No. ATCC 207014. This deposit was made under the provisions of the Budapest Treaty on the International Recognition of the Deposit of Microorganisms for the Purpose of Patent Procedure and the Regulation thereunder (Budapest Treaty). The deposit is provided as a convenience to those of skill in the art and is not an admission that a deposit is required under 35 U.S.C. §112. The deposited sequence, as well as the polypeptide encoded by the sequence, is incorporated herein by reference and controls in the event of any conflict, such as a sequencing error, with description in this application.

FGF-19 Polypeptides, Fragments, Variants, Derivatives and Analogs:

The invention further relates to FGF-19 polypeptides, and biologically active or diagnostically or therapeutically useful fragments, variants, derivatives and analogs thereof, and their use in modulating angiogenesis. In one embodiment, the FGF-19 polypeptide has the amino acid sequence of SEQ ID NO:2. In another embodiment, the FGF-19 polypeptide is a fragment, variant, derivative or analog of SEQ ID NO:2. The FGF-19 polypeptide, fragment, variant, derivative or analog is biologically active. A biologically active FGF-19 polypeptide, fragment, variant, derivative or analog refers to the molecule's ability to modulate angiogenesis such as, for example, by affecting endothelial tube formation, as described in, for example, the HUVEC assays described in Examples 3 & 4 (infra), or by affecting tumor cell growth or proliferation (e.g., see Examples 5 and 6). Alternatively, such polypeptides, fragments, variants, derivatives or analogs which have the desired immunogenicity or antigenicity can be used in immunoassays, for immunization, for inhibition of FGF-19 polypeptide activity, and the like. Similarly, FGF-19 fragments, variants, derivatives or analogs that retain, or alternatively lack or inhibit, a desired FGF-19 property of interest (e.g., inhibition of angiogenesis) can be used as inducers, or inhibitors of such property and its physiological correlates. A specific embodiment relates to an FGF-19 fragment that can be administered to a subject to inhibit angiogenesis. Fragments, variants, derivatives or analogs of FGF-19 can be tested for the desired activity by procedures known in the art, including but not limited to the assays described herein.

In another embodiment, an FGF-19 polypeptide, fragment, variant, derivative or analog has at least 10 contiguous amino acids. In other embodiments, the FGF-19 polypeptide, fragment, variant, derivative or analog consists of at least 20 or 50 contiguous amino acids. In another embodiment, the FGF-19 polypeptide, fragments, variants, derivatives or analogs are not larger than 35, 100 or even 200 amino acids. Fragments, variants, derivatives and analogs of FGF-19 polypeptide include but are not limited to those molecules comprising regions that are substantially similar to an FGF-19 polypeptide (e.g., in various embodiments, at least 60%, or 70%, or 80%, or 90%, or up to 95% identity over an amino acid sequence of identical size), or when compared to an aligned sequence in which the alignment is done by a computer sequence comparison/alignment program known in the art, or when the encoding nucleic acid is capable of hybridizing to an FGF-19 nucleic acid, under stringent, moderately stringent, or low stringency conditions.

FGF-19 polypeptide variants, derivatives or analogs can be made by altering FGF-19 sequences by substitutions, additions or deletions that provide for functionally equivalent molecules. FGF-19 polypeptide variants, derivatives or analogs include, but are not limited to, those containing as a primary amino acid sequence of all or part of the amino acid sequence of an FGF-19 polypeptide including altered sequences in which functionally equivalent amino acid residues (i.e., conservative substitutions) are substituted for residues within the sequence, resulting in a silent change. For example, one or more amino acid residues within the sequence can be substituted by another amino acid of a similar polarity, hydrophobicity or hydrophilicity, which acts as a functional equivalent, thereby resulting in a silent alteration. Substitutes for an amino acid within the sequence can be selected from other members of the class to which the amino acid belongs.

FGF-19 polypeptide variants, fragments, derivatives and analogs can be produced by various methods known in the art. The manipulations which result in their production can occur at the gene or protein level. For example, the cloned FGF-19 gene or cDNA sequence can be modified by any of numerous strategies known in the art (see, e.g., Sambrook et al., Molecular Cloning, A Laboratory Manual, 2d Ed., Cold Spring Harbor Laboratory Press, Cold Spring Harbor, N.Y. (1989)). The sequence can be cleaved at appropriate sites with restriction endonuclease(s), followed by further enzymatic modification if desired, isolated, and ligated in vitro. In the production of a nucleic acid encoding an FGF-19 polypeptide, or fragment, variant, derivative or analog thereof, the modified nucleic acid remains in the proper translational reading frame, so that the reading frame is not interrupted by translational stop signals or other signals which interfere with the synthesis. An FGF-19 nucleic acid can also be mutated in vitro or in vivo to create and/or destroy translation, initiation and/or termination sequences. The nucleic acid sequence encoding an FGF-19 polypeptide can also be mutated to create variations in coding regions and/or to form new restriction endonuclease sites or destroy preexisting ones and to facilitate further in vitro modification. Any technique for mutagenesis known in the art can be used, including but not limited to chemical mutagenesis, in vitro site-directed mutagenesis (Hutchinson et al., J. Biol. Chem. 253:6551 (1978)), the use of TAB® linkers (Pharmacia), and the like.

Manipulations of the FGF-19 polypeptide sequence can also be made at the protein level. Included within the scope of the invention are FGF-19 polypeptide variants, derivatives or analogs which are chemically modified during or after translation (e.g., by glycosylation, acetylation, phosphorylation, amidation, derivatization by known protecting/blocking groups, proteolytic cleavage, linkage to an antibody molecule or other cellular ligand, and the like). Any of numerous chemical modifications can be carried out by known techniques, including, but not limited to, specific chemical cleavage (e.g., by cyanogen bromide); enzymatic cleavage (e.g., by trypsin, chymotrypsin, papain, V8 protease, and the like); modification by, for example, NaBH₄, acetylation, formylation, oxidation and reduction, or metabolic synthesis in the presence of tunicamycin, and the like.

In addition, FGF-19 polypeptides, or fragments, variants, derivatives and analogs thereof can be chemically synthesized. For example, a peptide corresponding to a portion, or fragment, of an FGF-19 polypeptide, which comprises a desired domain, or which mediates a desired activity in vitro, can be synthesized by use of chemical synthetic methods using for example an automated peptide synthesizer. FGF-19 polypeptide analogs can be prepared, if desired, by introducing non-classical amino acids or chemical amino acid analogs as a substitution or addition into the FGF-19 polypeptide sequence. Non-classical amino acids include, but are not limited to, the D-isomers of the common amino acids, α-amino isobutyric acid, 4-aminobutyric acid, 2-amino butyric acid, γ-amino butyric acid, ε-Ahx, 6-amino hexanoic acid, 2-amino isobutyric acid, 3-amino propionic acid, omithine, norleucine, norvaline, hydroxyproline, sarcosine, citrulline, cysteic acid, t-butylglycine, t-butylalanine, phenylglycine, cyclohexylalanine, β-alanine, selenocysteine, fluoro-amino acids, designer amino acids such as β-methyl amino acids, C α-methyl amino acids, N α-methyl amino acids, and amino acid analogs in general. Furthermore, the amino acid can be D (dextrorotary) or L (levorotary).

In an embodiment, the FGF-19 polypeptide, or fragment, derivative or analog thereof is a chimeric, or fusion protein, comprising an FGF-19 polypeptide or variant, fragment, derivative or analog thereof (typically consisting of at least a domain or motif of the FGF-19 polypeptide, or at least 10 contiguous amino acids of the FGF-19 polypeptide) joined at its amino- or carboxy-terminus via a peptide bond to an amino acid sequence of a different protein. In one embodiment, such a chimeric protein is produced by recombinant expression of a nucleic acid encoding the protein. The chimeric product can be made by ligating the appropriate nucleic acid sequence, encoding the desired amino acid sequences to each other by methods known in the art, in the proper coding frame and expressing the chimeric product by methods commonly known in the art. Alternatively, the chimeric product can be made by protein synthetic techniques (e.g., by use of an automated peptide synthesizer).

Expression of FGF-19 Nucleic Acids or FGF-19 Polypeptides

In a further embodiment, host cells comprise a construct expressing an FGF-19 nucleic acid. Such a host cell can be a higher eukaryotic cell, such as a mammalian cell, a lower eukaryotic cell, such as a yeast cell, or a prokaryotic cell, such as a bacterial cell. Introduction of the construct into the host cell can be effected by calcium phosphate transfection, DEAE-dextran mediated transfection, or electroporation (Davis et al., Basic Methods in Molecular Biology, 2nd ed., Appleton and Lang, Paramount Publishing, East Norwalk, Conn. (1994)), calcium chloride-mediated transformation, lithium acetate-mediated transformation, and the like.

The constructs in host cells can be used in a conventional manner to produce the FGF-19 polypeptide, or fragment, variant, derivative or analog thereof. Cell-free translation systems can also be employed to produce such polypeptides using RNA's derived from the FGF-19 nucleic acid. Alternatively, the FGF-19 polypeptide, fragment, variant, derivative or analog thereof can be synthetically produced by conventional peptide synthesizers.

FGF-19 nucleic acids can be expressed in mammalian cells, yeast, bacteria, insect or other cells under the control of appropriate promoters. Representative expression vectors include plasmid, phage and/or viral vector sequences, such as those described by Sambrook et al., Molecular Cloning: A Laboratory Manual (Second Edition, Cold Spring Harbor, N.Y., (1989)). For example, suitable vectors include adenoviral vectors, retroviral vectors, including lentiviral vectors, vaccinia viral vectors, cytomegalovirus viral vectors, and baculovirus vectors (see, e.g., Knops et al., J. Biol. Chem. 266:7285 (1991)), and the like. DNA sequences derived from the SV40 viral genome, for example, SV40 origin, early promoter, enhancer, splice, and polyadenylation sites, can be used to provide the required nontranscribed genetic elements. Representative, useful vectors include pRc/CMV and pcDNA3 vectors (Invitrogen, San Diego, Calif.).

Promoters capable of directing the transcription of a nucleic acid can be inducible or constitutive promoters and include viral and cellular promoters. For expression in mammalian host cells, suitable viral promoters include the immediate early cytomegalovirus promoter (Boshart et al., Cell 41:521-30 (1985)) and the SV40 promoter (Subramani et al., Mol. Cell. Biol. 1:854-64 (1981)). Suitable cellular promoters for expression of nucleic acids in mammalian host cells include, but are not limited to the mouse metallothionien-1 promoter (Palmiter et al., U.S. Pat. No. 4,579,821), and tetracycline-responsive promoter (Gossen and Bujard, Proc. Natl. Acad. Sci. USA 89:5547-51 (1992); Pescini et al., Biochem. Biophys. Res. Comm. 202:1664-67 (1994)). Transcription termination signals are also typically located downstream of the coding sequence of interest. Suitable transcription termination signals include the early or late polyadenylation signals from SV40 (Kaufman and Sharp, Mol. Cell. Biol. 2:1304-19 (1982)), the polyadenylation signal from the Adenovirus 5 e1B region, and the human growth hormone gene terminator (DeNoto et al., Nucleic Acid. Res. 9:3719-30 (1981)).

Transcription of FGF-19 nucleic acids in mammalian cells is increased by inserting an enhancer sequence into the vector. Enhancers are cis-acting elements of DNA, usually about from 10 to 300 bp, that act on a promoter to increase its transcription. Examples include the SV40 enhancer on the late side of the replication origin (bp 100 to 270), a cytomegalovirus early promoter enhancer, a polyoma enhancer on the late side of the replication origin, and adenovirus enhancers.

Mammalian cells can be transfected by a number of methods including calcium phosphate precipitation (see, e.g., Wigler et al., Cell 14: 725 (1978); Corsaro and Pearson, Somatic Cell Genetics 7:603 (1981); Graham and Van der Eb, Virology 52: 456 (1973)); lipofection (see, e.g., Felgner et al., Proc. Natl. Acad. Sci. USA 84: 7413-17 (1987)) and microinjection and electroporation (see, e.g., Neumann et al., EMBO J. 1: 841-45 (1982)). Mammalian cells can be transduced with virus such as SV40, CMV and the like. In the case of viral vectors, cloned DNA molecules can be introduced by infection of susceptible cells with viral particles. Retroviral, including lentiviral, and adenoviral vectors are preferred for use in expressing FGF-19 nucleic acids in mammalian cells, particularly when FGF-19 nucleic acids or fragments, variants, derivatives or analogs thereof are used in methods of gene therapy.

Selectable markers are typically used to identify cells that contain the FGF-19 nucleic acids. Selectable markers are generally introduced into the cells along with the cloned DNA molecules and include genes that confer resistance to drugs, such as neomycin, hygromycin and methotrexate. Selectable markers can also complement auxotrophies in the host cell. Yet other selectable markers provide detectable signals, such as β-galactosidase or green fluorescent protein, to identify cells containing FGF-19 nucleic acids. Selectable markers can be amplifiable. Such amplifiable selectable markers can be used to amplify the number of sequences integrated into the host genome.

Various mammalian cell culture systems can be employed to express FGF-19 nucleic acids. Examples of mammalian expression systems include the COS-7 lines of monkey kidney fibroblasts (see, e.g., Gluzman, Cell 23:175 (1981)), and other cell lines capable of expressing a compatible vector, such as the C127, 3T3, CHO, HeLa, BHK, VERO, HeLa, MDCK, 293, WI38, HEK, HUVEC cell lines. Once established, such cell lines can be grown in culture. Methods for culturing human cells in vitro and for immortalizing cells are known to the skilled artisan.

For long-term, high-yield production of recombinant polypeptides, stable expression is preferred. For example, cell lines which stably express constructs containing the FGF-19 nucleic acids can be engineered. Rather than using expression vectors that contain viral origins of replication, host cells can be transformed with FGF-19 nucleic acids, by appropriate expression control elements (e.g., promoter and enhancer sequences, transcription terminators, polyadenylation sites, and the like), and a selectable marker. Following the introduction of such an expression vector into mammalian cells, engineered cells can be allowed to grow for 1-2 days in an enriched media, and then switched to a selective media. The selectable marker in the expression vector confers resistance to the selection and allows cells to stably integrate the vector into the chromosome and grow to form foci which in turn can be cloned and expanded into cell lines.

A number of selection systems can be used, including, but not limited, to the herpes simplex virus thymidine kinase (“tk”) (see, e.g., Wigler et al., Cell 11:223 (1977)), hypoxanthine-guanine phosphoribosyltransferase (“hprt”) (see, e.g., Szybalski et al., Proc. Natl. Acad. Sci. USA 48:2026 (1962)), and adenine phosphoribosyl-transferase genes (“aprt”) (see, e.g., Lowy et al., Cell 22: 817 (1980))and can be employed in tk⁻, hgprt⁻ or aprt⁻ cells, respectively. Antimetabolite resistance can also be used as the basis of selection for dihydrofolate reductase (“dhfr”), which confers resistance to methotrexate (Wigler et al., Proc. Natl. Acad. Sci. USA 77:3567(1980); O'Hare et al., FEBS Lett. 210:731 (1981)); gpt, which confers resistance to mycophenolic acid (Mulligan et al., Proc. Natl. Acad. Sci. USA 78:2072. (1981)); neomcyin, which confers resistance to the aminoglycoside G-418 (Colberre-Garapin et al., J. Mol. Biol. 150:1 (1981)); and hygromycin, which confers resistance to hygromycin (Santerre et al., Gene 30:147 (1984)).

FGF-19 nucleic acids can also be expressed in Saccharomyces cerevisiae, Schizosaccharomyces pombe, filamentous fungi, and other single and multicellular organisms that are amenable to transformation and/or transfection. Methods for expressing cloned genes in Saccharomyces cerevisiae are generally known in the art. (See, e.g., “Gene Expression Technology” In Methods in Enzymology, Vol. 185, Goeddel (ed.), Academic Press, San Diego, Calif. (1990); “Guide to Yeast Genetics and Molecular Biology” In Methods in Enzymology, Guthrie and Fink (eds.), Academic Press, San Diego, Calif. (1991)). Filamentous fungi (e.g., strains of Aspergillus) can also be used to express the FGF-19 nucleic acids. Methods for expressing heterologous genes and cDNAs in cultured mammalian cells and in E. coli are discussed in detail in Sambrook et al. (Molecular Cloning: A Laboratory Manual, Second Edition, Cold Spring Harbor, NY (1989)). As would be evident to one skilled in the art, one can express FGF-19 nucleic acids in other host cells such as avian, insect and plant cells using regulatory sequences, vectors and methods well established in the literature.

Recombinant expression vectors useful for expression in bacterial typically include origins of replication and selectable markers permitting transformation of the host cell (e.g., the ampicillin or tetracycline resistance genes of E. coli or the TRP1 or URA3 gene of S. cerevisiae), and a promoter derived from a highly-expressed gene to direct transcription of a downstream structural sequence. Such promoters can be derived from operons encoding glycolytic enzymes, such as 3-phosphoglycerate kinase (PGK), alpha factor, acid phosphatase, heat shock proteins, translation elongation factor, and the like. The heterologous FGF-19 nucleic acid is assembled in appropriate phase with translation initiation and termination sequences, and preferably, a leader sequence capable of directing secretion of translated protein into the periplasmic space or extracellular medium. Optionally, the heterologous sequence can encode a fusion protein including an N-terminal identification peptide imparting desired characteristics (e.g., stabilization or simplified purification of expressed recombinant product). Suitable prokaryotic hosts for transformation include Escherichia coli, Bacillus subtilis, Salmonella typhimurium and various species within the genera Pseudomonas, Streptomyces, and Staphylococcus, although others can also be employed as a routine matter of choice.

Useful expression vectors for bacterial use comprise a selectable marker and bacterial origin of replication derived from plasmids comprising genetic elements of the well-known cloning vector pBR322 (ATCC 37017). Other vectors include but are not limited to, pBLUESCRIPT vectors (Stratagene), PKK223-3 (Pharmacia Fine Chemicals, Uppsala, Sweden) and GEM1 (Promega Biotec, Madison, Wis.). These pBR322 “backbone” sections are combined with an appropriate promoter and the structural sequence to be expressed.

Useful expression vectors further comprise a fusion partner for ease in purifying a desired polypeptide or for producing soluble polypeptides. Examples of commercial fusion vectors include but are not limited to pET32a (Novagen, Madison, Wis.), pGEX-4T-2 (Pharmacia) and pCYB3 (New England Biolabs, Beverly, Mass.). Expression vectors which avoid the use of fusion partners can also be constructed particularly for high level expression of FGF-19 polypeptides, or fragments, variants, derivatives or analogs thereof in bacterial cells. For example, vectors can be made to optimize translational coupling, as described by Pilot-Matias et al. (Gene 128:219-225 (1993)). Alternatively, an FGF-19 nucleic acid can be co-expressed with a separate accessory plasmid which itself encodes a protein or peptide that aids in solubilizing an FGF-19 polypeptide of interest. (See, e.g., Makrides, Microbiological Reviews 60:512 (1996)).

Following transformation of a suitable host strain and growth of the host strain to an appropriate cell density, the selected promoter is derepressed by appropriate means (e.g., temperature shift or chemical induction), and cells are cultured for an additional period. Cells are typically harvested by centrifugation, disrupted by physical or chemical means, and the resulting crude extract retained for further purification. Microbial cells employed in expression of proteins can be disrupted by any convenient method, including freeze-thaw cycling, sonication, mechanical disruption, or use of cell lysing agents; such methods are well-known to the ordinary artisan.

FGF-19 polypeptides can be isolated using a number of established methods, such as affinity chromatography using anti-FGF-19 antibodies coupled to a solid support and sequence-specific chromatography as described by Lobanenkov et al. (Oncogene 5:1743-53 (1990)) and using antibodies against an epitope-tagged FGF-19 polypeptide (e.g., anti-HIS₄, myc, FLAG, and the like). Additional isolation methods include purification means such as liquid chromatography, high pressure liquid chromatography, FPLC, gradient centrifugation and gel electrophoresis, among others. Methods of protein purification are known in the art and can be applied to the purification of recombinant polypeptides described herein. (See generally, Scopes, Protein Purification, Springer-Verlag, NY (1982)).

Anti-FGF-19 Antibodies

In another embodiment, the invention provides anti-FGF-19 antibodies for use in modulating angiogenesis. Such antibodies can bind to FGF-19 polypeptides, or fragments, variants, derivatives or analogs thereof. FGF-19 polypeptides can be used to raise antisera or monoclonal antibodies following, for example, the method of Kohler and Milstein (Nature 256:495 (1975)). Such monoclonal antibodies can then form the basis for a treatment, therapeutic use, or diagnostic test.

The production of non-human antisera or monoclonal antibodies (e.g., murine, lagormorpha, porcine or equine) can be accomplished by, for example, immunizing an animal with FGF-19 polypeptides, fragments, variants, derivatives or analogs, with or without an adjuvant. For the production of monoclonal antibodies, antibody producing cells are obtained from immunized animals, immortalized and screened, or screened first for the production of the antibody that binds to the antigen, and then immortalized. It can be desirable to transfer the antigen binding regions (e.g., F(ab′), F(ab′)₂, Fv, or hypervariable regions) of non-human antibodies into the framework of a human antibody by recombinant DNA techniques to produce a substantially human molecule. Methods for producing such “humanized” molecules are generally well known and described in, for example, U.S. Pat. Nos. 4,816,567; 4,816,397; 5,693,762; and 5,712,120; International Patent Publication WO 87/02671 and WO 90/00616; and European Patent Publication 0,239,400; the disclosures of which are incorporated by reference herein). Alternatively, a human monoclonal antibody or portions thereof can be identified by first screening a human B-cell cDNA library for DNA molecules that encode antibodies that specifically bind to an FGF-19 polypeptide according to the method generally set forth by Huse et al. (Science 246:1275-81 (1989)). The DNA molecule can then be cloned and amplified to obtain sequences that encode the antibody (or binding domain) of the desired specificity. Phage display technology offers another technique for selecting antibodies that bind to FGF-19 polypeptides. (See, e.g., International Patent Publications WO 91/17271 and WO 92/01047, and Huse et al., supra).

Antibodies can also be produced by genetic immunization using expression vectors to direct the expression of FGF-19 polypeptides. Particle bombardment-mediated gene transfer (Tang et al., Nature 356:152-54 (1992); Eisenbaum et al., DNA & Cell Biol. 12:791-97 (1993); Johnston and Tang, Meth. Cell Biol. 43 Pt.A:353-65 (1994); Vahlsing et al., J. Immun. Meth. 175:11-22 (1994)) and retroviral gene transfer (Wang et al., DNA & Cell Biol. 12:799-805 (1993); Stover, Curr. Opin. Immunol. 6:568-71 (1994); Laube et al., Human Gene Ther. 5:853-62 (1994)) have been used to generate specific antibody responses to proteins encoded by transferred genes. These methods permit the production of antibodies without requiring protein purification. Such methods can be used to produce panels of antibodies specific to FGF-19 polypeptides. Monoclonal antibodies can also be generated using these methods.

Antibodies against FGF-19 polypeptides can be used as reagents to detect FGF-19 polypeptides in biological samples, such as tumor biopsy samples, tissue and organ sections, peripheral blood cells, and the like. Such antibodies can also be used in immunoassays to detect and/or quantitate FGF-19 polypeptide levels. Immunoassays suitable for use in the present invention include, but are not limited to, enzyme-linked immunosorbant assays, immunoblots, inhibition or competition reactions, sandwich assays, radioimmunoprecipitation, and the like. (See, e.g., U.S. Pat. Nos. 4,642,285; 4,376,110; 4,016,043; 3,879,262; 3,852,157; 3,850,752; 3,839,153; 3,791,932; and Harlow and Lane, Antibodies, A Laboratory Manual, Cold Spring Harbor Publications, NY (1988)).

In one assay, FGF-19 polypeptides are identified and/or quantified using labeled antibodies, preferably labeled monoclonal antibodies. The antibodies are reacted with tissues or cells, and then the tissues or cells are examined to determine whether the antibodies specifically bound to the target FGF-19 polypeptide. Such assays are typically performed under conditions conducive to immune complex formation. Unlabeled primary antibody can be used in combination with labels that are reactive with primary antibody to detect the FGF-19 polypeptide. For example, the primary antibody can be detected indirectly by a labeled secondary antibody made to specifically detect the primary antibody. Alternatively, the anti-FGF-19 antibody can be directly labeled. A wide variety of labels can be employed, such as radionuclides, particles (e.g., gold, ferritin, magnetic particles and red blood cells), fluorophores, chemiluminescers, enzymes, enzyme substrates, enzyme cofactors, enzyme inhibitors, ligands (particularly haptens), and the like.

The anti-FGF-19 polypeptides can be used in a diagnostic assay for detecting levels of polypeptides of the present invention, for example, in various tissues, since an under-expression of the proteins compared to normal control tissue samples may detect the presence of abnormal angiogenesis, for example, a tumor. Assays used to detect levels of protein in a sample derived from a host are well-known to those of skill in the art and include radioimmunoassays, competitive-binding assays, Western blot analyses, ELISA assays and “sandwich” type assays. Diagnostic assays also include the detection of polynucleotides which code for the polypeptides of the present invention.

Methods of Using FGF-19 Nucleic Acids and/or FGF-19 Polypeptides:

In another embodiment, methods and compositions are provided for the administration of an FGF-19 compound to modulate angiogenesis. FGF-19 compounds include, but are not limited to, FGF-19 polypeptides, fragments, variants, derivatives and analogs thereof, as described herein. Such FGF-19 compounds can further include FGF-19 nucleic acids encoding FGF-19 polypeptide, fragments or variants, as described herein, and FGF-19 antisense nucleic acids, as more fully described below. Disorders involving angiogenesis, such as unwanted angiogenesis, use an FGF-19 compound that inhibits angiogenesis, such as the administration of FGF-19 polypeptides, fragments, variants, derivatives and/or analogs thereof, and/or FGF-19 nucleic acids. Similarly, disorders in which angiogenesis is deficient or is desired can be treated by administration an FGF-19 antisense nucleic acid, or an FGF-19 polypeptide, a fragment, variant, derivative or analog thereof that inhibits FGF-19 function. In another embodiment, FGF-19 compounds include antibodies, such as polyclonal, monoclonal and humanized antibodies.

The compounds can be administered therapeutically or prophylactically. They can be contacted with the host cell in vivo, ex vivo, or in vitro, in an effective amount, as demonstrated by the following examples.

Gene Therapy

FGF-19 nucleic acids coding for FGF-19 polypeptides of the present invention, can be used in a process of gene therapy. Gene therapy refers to the process of providing for the expression of nucleic acid sequences of exogenous origin in a subject for the treatment of a disease or clinical condition within that subject. Such gene therapy can be involved in the treatment of a disease or clinical condition which can include, but is not limited to, cancer, wound healing, diabetic retinopathies, macular degeneration, cardiovascular diseases, and clinical conditions involving angiogenesis in the reproductive system, including regulation of placental vascularization or use as an abortifacient. Delivery of the nucleic acid into a subject can be either direct, in which case the patient is directly exposed to the nucleic acid or nucleic acid-carrying vector, or indirect, in which case cells are first transformed with the nucleic acid in vitro, then transplanted into the patient. These two approaches are known, respectively, as in vivo or ex vivo gene therapy. For example, FGF-19 polypeptide, or a fragment or variant thereof, can be recombinantly expressed by engineering cells with a polynucleotide (DNA or RNA) coding for the polypeptide, fragment or variant ex vivo, the engineered cells are then provided to a patient to be treated with the polypeptide. Cells can also be engineered by procedures known in the art by use of a retroviral/lentiviral particle containing RNA encoding the FGF-19 polypeptide, fragment of variant. Exemplary methods are described below.

For general reviews of the methods of gene therapy, see Goldspiel et al. (Clinical Pharmacy 12:488-505 (1993)); Wu and Wu (Biotherapy 3:87-95 (1991)); Tolstoshev (Ann. Rev. Pharmacol. Toxicol. 32:573-596 (1993)); Mulligan (Science 260:926-932 (1993)); Morgan and Anderson (Ann. Rev. Biochem. 62:191-217 (1993)); and May (TIBTECH 11:155-215 (1993)). Methods commonly known in the art of recombinant DNA technology that can be used include those described in Ausubel et al. (Current Protocols in Molecular Biology, John Wiley & Sons, NY (1993)); Kriegler (Gene Transfer and Expression, A Laboratory Manual, Stockton Press, NY (1990)); and U.S. Pat. Nos. 6,077,663; 6,077,835; 6077,705; and 6,075,012. Methods using other exogenous sequences for gene therapy are also applicable to gene therapy using FGF-19 nucleic acids. (See, e.g., U.S. Pat. No. 6,066,624).

Several methods for transferring potentially therapeutic genes to defined cell populations are known. (See, e.g., Mulligan, Science 260:926-31 ((1993).) These methods include:

1) Direct gene transfer. (See, e.g., Wolff et al., Science 247:1465-68 (1990)).

2) Liposome-mediated DNA transfer. (See, e.g., Caplen et al., Nature Med. 3:39-46 (1995); Crystal, Nature Med. 1:15-17 (1995); Gao and Huang, Biochem. Biophys. Res. Comm. 179:280-85 (1991)).

3) Retrovirus-mediated DNA transfer. (See, e.g., Kay et al., Science, 262:117-19 (1993); Anderson, Science 256:808-13 (1992)). Retroviruses from which the retroviral plasmid vectors hereinabove mentioned can be derived include lentiviruses. They further include, but are not limited to, Moloney Murine Leukemia Virus, spleen necrosis virus, retroviruses such as Rous Sarcoma Virus, Harvey Sarcoma Virus, avian leukosis virus, gibbon ape leukemia virus, human immunodeficiency virus, Myeloproliferative Sarcoma Virus, and mammary tumor virus. In one embodiment, the retroviral plasmid vector is derived from Moloney Murine Leukemia Virus. Examples illustrating the use of retroviral vectors in gene therapy further include the following: Clowes et al. (J. Clin. Invest. 93:644-651 (1994)); Kiem et al. (Blood 83:1467-1473 (1994)); Salmons and Gunzberg (Human Gene Therapy 4:129-141 (1993)); and Grossman and Wilson (Curr. Opin. in Genetics and Devel. 3:110-114 (1993)).

4) DNA Virus-mediated DNA transfer. Such DNA viruses include adenoviruses (preferably Ad-2 or Ad-5 based vectors), herpes viruses (preferably herpes simplex virus based vectors), and parvoviruses (preferably “defective” or non-autonomous parvovirus based vectors, more preferably adeno-associated virus based vectors, most preferably AAV-2 based vectors). (See, e.g., Ali et al., Gene Therapy 1:367-84 (1994); U.S. Pat. Nos. 4,797,368 and 5,139,941, the disclosures of which are incorporated herein by reference.) Adenoviruses have the advantage that they have a broad host range, can infect quiescent or terminally differentiated cells, such as neurons or hepatocytes, and appear essentially non-oncogenic. Adenoviruses do not appear to integrate into the host genome. Because they exist extrachromosomally, the risk of insertional mutagenesis is greatly reduced. Adeno-associated viruses exhibit similar advantages as adenoviral-based vectors. However, AAVs exhibit site-specific integration on human chromosome 19.

Kozarsky and Wilson (Current Opinion in Genetics and Development 3:499-503 (1993)) present a review of adenovirus-based gene therapy. Bout et al. (Human Gene Therapy 5:3-10 (1994)) demonstrated the use of adenovirus vectors to transfer genes to the respiratory epithelia of rhesus monkeys. Herman et al. (Human Gene Therapy 10:1239-1249 (1999)) describe the intraprostatic injection of a replication-deficient adenovirus containing the herpes simplex thymidine kinase gene into human prostate, followed by intravenous administration of the prodrug ganciclovir in a phase I clinical trial. Other instances of the use of adenoviruses in gene therapy can be found in Rosenfeld et al. (Science 252:431-434 (1991)); Rosenfeld et al. (Cell 68:143-155 (1992)); Mastrangeli et al. (J. Clin. Invest. 91:225-234 (1993)); and Thompson (Oncol. Res. 11:1-8 (1999)).

The choice of a particular vector system for transferring the gene of interest will depend on a variety of factors. One important factor is the nature of the target cell population. Although retroviral vectors have been extensively studied and used in a number of gene therapy applications, these vectors are generally unsuited for infecting non-dividing cells. In addition, retroviruses have the potential for oncogenicity. However, recent developments in the field of lentiviral vectors may circumvent some of these limitations. (See Naldini et al., Science 272:263-7 (1996).)

Gene therapy with DNA encoding a polypeptide of the present invention is provided to a subject (e.g., a patient or mammal) in need thereof, concurrent with, or immediately after diagnosis. The skilled artisan will appreciate that any suitable gene therapy vector containing DNA encoding a polypeptide of the present invention can be used in accordance with the present invention. The techniques for constructing such a vector are known. (See, e.g., Anderson, Nature 392 25-30 (1998); Verma, Nature 389 239-42 (1998)). Introduction of the vector to the target site can be accomplished using known techniques.

In one embodiment, FGF-19 nucleic acid is inserted into an expression vector. The FGF-19 nucleic acids encode an FGF-19 polypeptide, fragment, variant, derivative or chimeric protein. In particular, such an expression vector construct typically comprises a promoter operably linked to an FGF-19 nucleic acid (e.g., cDNA or a portion of the coding region), the promoter being inducible or constitutive, and, optionally, tissue-specific.

In another embodiment, if an endogenous FGF-19 nucleic acid is defective, the defective sequences can be replaced by exogenous FGF-19 coding sequences and any other desired sequences that are flanked by regions that promote homologous recombination at a desired site in the genome, thus providing for intrachromosomal expression of the exogenous FGF-19 nucleic acid. (See e.g., Koller and Smithies, Proc. Natl. Acad. Sci. USA 86:8932-8935 (1989); Zijlstra et al., Nature 342:435-438 (1989)); U.S. Pat. Nos. 5,631,153; 5,627,059; 5,487,992; and 5,464,764.).

Nucleic acids can also be administered in linkage to a peptide which is known to enter the nucleus, by administering the nucleic acid in linkage to a ligand subject to receptor-mediated endocytosis (see, e.g., Wu and Wu, J. Biol. Chem. 262:4429-4432 (1987)), which can be used to target cell types specifically expressing the receptors, and the like. In another embodiment, a nucleic acid-ligand complex can be formed in which the ligand comprises a fusogenic viral peptide to disrupt endosomes, allowing the nucleic acid to avoid lysosomal degradation.

In yet another embodiment, the nucleic acid can be targeted in vivo for cell specific uptake and expression, by targeting a specific receptor (see, e.g., International Patent Publications WO 92/06180; WO 92/22635; WO 92/20316; WO 93/14188, and WO 93/20221). Alternatively, the nucleic acid can be introduced intracellularly and incorporated within host cell DNA for expression by homologous recombination. (See, e.g., Koller and Smithies, Proc. Natl. Acad. Sci. USA 86:8932-8935 (1989); Zijistra et al., Nature 342:435-438 (1989); U.S. Pat. Nos. 5,631,153; 5,627,059; 5,487,992; and 5,464,764).)

In a specific embodiment, a viral vector is used that contains an FGF-19 nucleic acid. For example, a retroviral vector can be used (see Miller et al., Meth. Enzymol. 17:581-599 (1993)). These retroviral vectors have been modified to delete retroviral sequences that are not necessary for packaging of the viral genome and integration into host cell DNA. The FGF-19 nucleic acid to be used in gene therapy is cloned into the vector, which facilitates delivery of the gene into a patient. More detail about retroviral vectors can be found in Boesen et al. (Biotherapy 6:291-302 (1994)), which describes the use of a retroviral vector to deliver the mdrl gene to hematopoietic stem cells in order to make the stem cells more resistant to chemotherapy.

Other approaches to gene therapy involve transferring a gene to cells in tissue culture by methods such as electroporation, lipofection, calcium phosphate mediated transfection, or viral infection. Typically, the method includes the transfer of a selectable marker to the cells. The cells are then placed under selection to isolate those cells that have taken up and are expressing the transferred gene. The selected cells are then delivered to a patient.

Numerous techniques are known in the art for the introduction of foreign genes into cells (see, e.g., Loeffler and Behr, Meth. Enzymol. 217:599-618 (1993); Cohen et al., Meth. Enzymol. 217:618-644 (1993); Cline, Pharmac. Ther. 29:69-92 (1985)) and can be used in accordance with the present invention. The technique typically provides for the stable transfer of the nucleic acid to the cell, so that the nucleic acid is expressible by the cell and is heritable and expressible by its cell progeny.

The resulting recombinant cells can be delivered to a patient by various methods known in the art. Typically, cells are injected subcutaneously. Alternatively, recombinant skin cells can be applied as a skin graft onto the patient. Recombinant blood cells (e.g., hematopoietic stem or progenitor cells) are typically administered intravenously. The amount of cells envisioned for use depends on the desired effect, the patient's condition, and the like, and can be determined by one skilled in the art.

Cells into which a nucleic acid can be introduced for purposes of gene therapy encompass any desired, available cell type, and include but are not limited to endothelial cells, prostate cells, epithelial cells, keratinocytes, fibroblasts, muscle cells, hepatocytes, blood cells (such as T lymphocytes, B lymphocytes, monocytes, macrophages, neutrophils, eosinophils, megakaryocytes and granulocytes), various stem or progenitor cells (in particular, hematopoietic stem or progenitor cells, such as those obtained from bone marrow, umbilical cord blood, peripheral blood, fetal liver, and the like). The cells used for gene therapy generally are autologous to the patient, but heterologous cells that can be typed for compatibility with the patient can be used.

Administration of FGF-19 Polypeptides, or Fragments, Variants, Derivatives or Analogs Thereof:

The invention provides methods for the administration to a subject of an effective amount of an FGF-19 compound. For example, unwanted angiogenesis can be treated or prevented by administration of an effective amount of FGF-19 polypeptide, fragment, variant, derivative or analog thereof. In one embodiment, such polypeptides are administered therapeutically (including prophylactically) in diseases or clinical condition involving increased (relative to normal or desired) angiogenesis, to thereby inhibit angiogenesis. In another embodiment, such polypeptides are administered therapeutically (including prophylactically) in diseases or clinical conditions where angiogenesis may be relevant to the causation or treatment of the disease or clinical condition in order to inhibit the disease or clinical condition. The diseases or clinical conditions of the present invention include but are not limited to, cancer, wound healing, tumor formation, diabetic retinopathies, macular degeneration, cardiovascular diseases, and the like.

Typically, the FGF-19 compound is substantially purified prior to formulation. The subject can be an animal, including but not limited to, cows, pigs, horses, chickens, cats, dogs, and the like, and is typically a mammal, and in a particular embodiment human. In another specific embodiment, a non-human mammal is the subject. Formulations and methods of administration that can be employed when the FGF-19 compound comprises a nucleic acid are described above; additional appropriate formulations and routes of administration can be selected from among those described below.

Various delivery systems are known and can be used to administer an FGF-19 compound, such as, for example, encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of an expression vector comprising an FGF-19 nucleic acid as part of a retroviral or other vector, and the like. Methods of introduction include but are not limited to, intradermal, intramuscular, intraperitoneal, intravenous, subcutaneous, intranasal, epidural and oral routes. The compounds can be administered by any convenient route, for example, by infusion or bolus injection, by absorption through epithelial or mucocutaneous linings (e.g., oral mucosa, rectal and intestinal mucosa) and the like, and can be administered together with other biologically active agents. Administration can be systemic or local.

In a specific embodiment, it can be desirable to administer an FGF-19 compound locally to the area in need of treatment; this administration can be achieved by, for example, and not by way of limitation, local infusion during surgery, topical application (e.g., in conjunction with a wound dressing after surgery), by injection, by means of a catheter, by means of a suppository, or by means of an implant, the implant being of a porous, non-porous, or gelatinous material, including membranes such as sialastic membranes, or fibers. In one embodiment, administration can be by direct injection at the site (or former site) of a malignant tumor or neoplastic or pre-neoplastic tissue.

In another embodiment, the FGF-19 compound can be delivered in a vesicle, in particular a liposome (see Langer, Science 249:1527-1533 (1990); Treat et al., In Liposomes in the Therapy of Infectious Disease and Cancer, Lopez-Berestein and Fidler (eds.), Liss, N.Y., pp. 353-365 (1989); Lopez-Berestein, supra, pp. 317-327); U.S. Pat. Nos. 6,077,663 and 6,071,533).

In yet another embodiment, the FGF-19 compound can be delivered in a controlled release system. In one embodiment, a pump can be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see, e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J. Macromol. Sci. Rev. Macromol. Chem. 23:61 (1983); Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, thus requiring only a fraction of the systemic dose (see, e.g., Goodson, in Medical Applications of Controlled Release, supra, Vol. 2, pp. 115-138 (1984)). Other controlled release systems are discussed in, for example, the review by Langer (Science 249:1527-1533 (1990)).

The present invention also provides pharmaceutical compositions for administering FGF-19 compounds. Such compositions comprise a therapeutically effective amount of an FGF-19 compound and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more typically in humans. The term “carrier” refers to a diluent, adjuvant, excipient, stabilizer, or vehicle with which the FGF-19 compound is formulated for administration.

A pharmaceutically acceptable carrier can contain a physiologically acceptable compound that acts, for example, to stabilize the composition or to increase or decrease the absorption of the agent. A physiologically acceptable compound can include, for example, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents or preservatives, which are particularly useful for preventing the growth or action of microorganisms. Carriers further include sterile liquids, such as water and oils, including those of petroleum, animal, vegetable or synthetic origin, such as peanut oil, soybean oil, mineral oil, sesame oil, and the like. Water is a typical carrier when the pharmaceutical composition is administered intravenously. Saline solutions and aqueous dextrose and glycerol solutions can also be employed as liquid carriers, particularly for injectable solutions. Various preservatives are well known and include, for example, phenol and ascorbic acid. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. One skilled in the art would know that the choice of a pharmaceutically acceptable carrier, including a physiologically acceptable compound, depends, for example, on the route of administration of the polypeptide and on the particular physio-chemical characteristics of the specific polypeptide. For example, a physiologically acceptable carrier such as aluminum monosterate or gelatin is particularly useful as a delaying agent, which prolongs the rate of absorption of a pharmaceutical composition administered to a subject. Further examples of carriers, stabilizers or adjutants can be found in Martin, Remington's Pharm. Sci., 15th Ed. (Mack Publ. Co., Easton, 1975), incorporated herein by reference. The pharmaceutical composition also can be incorporated, if desired, into liposomes, microspheres or other polymer matrices (see Gregoriadis, Liposome Technology, Vol. 1 (CRC Press, Boca Raton, Fla. 1984), which is incorporated herein by reference). Liposomes, for example, which consist of phospholipids or other lipids, are nontoxic, physiologically acceptable and metabolizable carriers that are relatively simple to make and administer.

When practiced in vivo, methods of administering a pharmaceutical composition containing the vector of this invention, are well known in the art and include but are not limited to, administration orally, intra-tumorally, intravenously, intramuscularly or intraperitoneal. Administration can be effected continuously or intermittently and will vary with the subject and the condition to be treated, for example, as is the case with other therapeutic compositions (see Landmann et al., J. Interferon Res. 12:103-111 (1992); Aulitzky et al., Eur. J. Cancer 27:462-67 (1991); Lantz et al., Cytokine 2:402-06 (1990); Supersaxo et al., Pharm. Res. 5:472-76 (1988); Demetri et al., J. Clin. Oncol. 7:1545-53 (1989); and LeMaistre et al., Lancet 337:1124-25 (1991)).

Pharmaceutical compositions can take the form of solutions, suspensions, emulsion, tablets, pills, capsules, powders, sustained-release formulations, and the like. The composition can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, and the like.

Pharmaceutical compositions will contain a therapeutically effective amount of the FGF-19 compound, typically in purified form, together with a suitable amount of carrier so as to provide a formulation proper for administration to the patient. The formulation should suit the mode of administration.

In one embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition can also include a solubilizing agent and a local anesthetic to ease pain at the site of the injection. Generally, the ingredients are supplied either separately or mixed together in unit dosage form. For example, as a dry lyophilized powder or water-free concentrate in a hermetically sealed container such as an ampoule or sachette indicating the quantity of FGF-19 compound. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

The FGF-19 compounds can be formulated as neutral or salt forms. Pharmaceutically acceptable salts include those formed with free amino groups such as those derived from hydrochloric, phosphoric, acetic, oxalic, tartaric acids, and the like, and those formed with free carboxyl groups such as those derived from sodium, potassium, ammonium, calcium, ferric hydroxides, isopropylamine, triethylamine, 2-ethylamino ethanol, histidine, procaine, and the like.

The amount of the FGF-19 compound that will be effective in the treatment of a particular disorder or condition as indicated by modulation of angiogenesis will depend on the nature of the disorder or condition, and can be determined by standard clinical techniques. In addition, in vitro assays can optionally be employed to help identify optimal dosage ranges. The precise dose of the FGF-19 compound to be employed in the formulation will also depend on the route of administration, and the seriousness of the disease or disorder, and should be decided according to the judgment of the practitioner and each patient's circumstances. Suitable dosage ranges for intravenous administration are generally about 20-500 micrograms of active FGF-19 compound per kilogram body weight. Suitable dosage ranges for intranasal administration are generally about 0.01 pg/kg body weight to 1 mg/kg body weight. Effective doses can be extrapolated from dose response curves derived from in vitro or animal model test systems. Suppositories generally contain active ingredient in the range of 0.5% to 10% by weight; oral formulations typically contain 10% to 95% active ingredient.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

Treatment of Angiogenesis-Related Diseases

In another embodiment, FGF-19 compounds, such as FGF-19 polypeptides, or fragments, variants, derivatives or analogs thereof, or FGF-19 nucleic acids encoding such polypeptides, can be used to treat diseases or clinical conditions that may be related to angiogenesis. Without intending to be bound by any particular theory, it is believed that the initiation and/or progression of many diseases is dependent on angiogenesis. For example, tumor formation is closely associated with angiogenesis. Such tumors include solid tumors, such as rhabdomyosarcomas, retinoblastoma, Ewing sarcoma, neuroblastoma, and osteosarcoma, and benign tumors, such as acoustic neuroma, neurofibroma, trachoma and pyogenic granulomas. Thus, tumor formation or progression can be treated by inhibiting angiogenesis; administering FGF-19 polypeptides, fragments, variants, derivatives and analogs, or FGF-19 nucleic acids, to the tumor will inhibit further tumor growth and/or progression. Similarly, cells expressing recombinant FGF-19 polypeptides, fragments or variants can be used. Alternatively, decreased angiogenesis is associated with cardiovascular disease. Thus, FGF-19 compounds that increase angiogenesis can be used to treat angiogenesis. Any of the methodologies described above can be applied to the treatment of such angiogenesis-related diseases.

Other diseases or clinical conditions involving unwanted angiogenesis can also be treated in a similar manner. Such other diseases or conditions include, but are not limited to, ocular neovascular disease, age-related macular degeneration, diabetic retinopathy, corneal graft rejection, neovascular glaucoma and retrolental fibroplasia, epidemic keratoconjunctivitis, Vitamin A deficiency, contact lens overwear, atopic keratitis, superior limbic keratitis, pterygium, keratitis sicca, Sjogren's syndrome, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi sarcoma, Mooren ulcer, Terrien's marginal degeneration, mariginal keratolysis, rheumatoid arthritis, systemic lupus, polyarthritis, Wegener's sarcoidosis, Scleritis, Steven's Johnson disease, periphigoid radial keratotomy, corneal graph rejection, sickle cell anemia, sarcoidosis, syphilis, pseudoxanthoma elasticum, Pagets disease, vein occlusion, artery occlusion, carotid obstructive disease, chronic uveitis/vitreitis, Lyme's disease, systemic lupus erythematosis, retinopathy of prematurity, Eales disease, Bechets disease, infections causing a retinitis or choroiditis, presumed ocular histoplasmosis, Bests disease, myopia, optic pits, Stargart's disease, pars planitis, chronic retinal detachment, hyperviscosity syndromes, toxoplasmosis, trauma and post-laser complications. Other diseases include those associated with rubeosis, abnormal proliferation of fibrovascular or fibrous tissue, rheumatoid arthritis, osteoarthritis, ulcerative colitis, Crohn's disease, Bartonellosis, atherosclerosis, hemangioma, Osler-Weber-Rendu disease, hereditary hemorrhagic telangiectasia, and leukemia.

Antisense Regulation of FGF-19 Expression:

In a specific embodiment, FGF-19 function is inhibited by use of FGF-19 antisense nucleic acids. The present invention provides for the administration of nucleic acids of at least six nucleotides that are antisense to a gene or cDNA encoding FGF-19 polypeptide or a fragment or variant thereof to inhibit the function of FGF-19 polypeptide. An FGF-19 “antisense” nucleic acid as used herein refers to a nucleic acid that hybridizes to a portion of an FGF-19 RNA (typically mRNA) by virtue of some sequence complementarity. The antisense nucleic acid can be complementary to a coding and/or noncoding region of an FGF-19 mRNA. Absolute complementarity, although typical, is not required, however. A sequence “complementary to at least a portion of an RNA,” as referred to herein, means a sequence having sufficient complementarity to be able to hybridize with the RNA, forming a stable duplex. In the case of double-stranded FGF-19 antisense nucleic acids, a single strand of the duplex DNA can be tested, or triplex formation can be assayed. The ability to hybridize will depend on both the degree of complementarity and the length of the antisense nucleic acid. Generally, the longer the hybridizing nucleic acid, the more base mismatches it can contain and still form a stable duplex (or triplex, as the case may be). One skilled in the art can ascertain a tolerable degree of mismatch by use of standard procedures to determine the melting point of the hybridized complex.

Such antisense nucleic acids have utility as agents that inhibit FGF-19 function, and can be used in the treatment or prevention of diseases or clinical conditions, as described supra. The antisense nucleic acids of the invention can be oligonucleotides that are double-stranded or single-stranded, RNA or DNA, or a derivative thereof, which can be directly administered to a cell, or which can be produced intracellularly by transcription of exogenous, introduced nucleic acid sequences.

In a specific embodiment, the FGF-19 antisense nucleic acid provided by the instant invention can be used to prevent angiogenesis. The invention further provides pharmaceutical compositions comprising an effective amount of the FGF-19 antisense nucleic acids of the invention in a pharmaceutically acceptable carrier, as described supra. In another embodiment, the invention is directed to methods for inhibiting the expression of an FGF-19 nucleic acid sequence in a eukaryotic cell comprising providing the cell with an effective amount of a composition comprising an FGF-19 antisense nucleic acid of the invention. FGF-19 antisense nucleic acids and their uses are described in detail below.

The FGF-19 antisense nucleic acids are of at least six nucleotides and are typically oligonucleotides (ranging from 6 to about 50 nucleotides or more). In specific aspects, the oligonucleotide is at least 10 nucleotides, at least 15 nucleotides, at least 100 nucleotides, or can be at least 200 nucleotides. The oligonucleotides can be DNA or RNA or chimeric mixtures or derivatives thereof, and can be single-stranded or double-stranded. A derivative can be modified at the base moiety, sugar moiety, or phosphate backbone, as described below. The derivative can include other appending groups such as peptides, or agents facilitating transport across the cell membrane (see, e.g., Letsinger et al., Proc. Natl. Acad. Sci. USA 86:6553-56 (1989); Lemaitre et al., Proc. Natl. Acad. Sci. USA 84:648-52 (1987); International Patent Publication WO 88/09810) or blood-brain barrier (see, e.g., International Patent Publication WO 89/10134), hybridization-triggered cleavage agents (see, e.g., Krol et al., BioTechniques 6:958-76 (1988)) or intercalating agents (see, e.g., Zon, Pharm. Res. 5:539-49 (1988)).

In one embodiment of the invention, a FGF-19 antisense oligonucleotide is provided, typically as single-stranded DNA. The oligonucleotide can be modified at any position on its structure with substituents generally known in the art. The FGF-19 antisense oligonucleotide can comprise at least one modified base moiety, such as, for example, 5-fluorouracil, 5-bromouracil, 5-chlorouracil, 5-iodouracil, hypoxanthine, xanthine, 4-acetylcytosine, 5-(carboxy-hydroxylmethyl) uracil, 5-carboxymethylaminomethyl-2-thiouridine, 5-carboxymethylamino-methyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v) , pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, 2,6-diaminopurine, and the like. In another embodiment, the oligonucleotide comprises at least one modified sugar moiety, such as, for example, arabinose, 2-fluoroarabinose, xylulose, and hexose.

In yet another embodiment, the oligonucleotide comprises at least one modified phosphate backbone, such as, for example, a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the oligonucleotide is an α-anomeric oligonucleotide. An α-anomeric oligonucleotide forms specific double-stranded hybrids with complementary RNA in which, contrary to the usual β-units, the strands run parallel to each other (see Gautier et al., Nucl. Acids Res. 15:6625-41 (1987)). The oligonucleotide can be conjugated to another molecule (e.g., a peptide, hybridization triggered cross-linking agent, transport agent, hybridization-triggered cleavage agent, and the like).

Oligonucleotides of the invention can be synthesized by standard methods known in the art (e.g., by use of a commercially available automated DNA synthesizer). As examples, phosphorothioate oligonucleotides can be synthesized by the method of Stein et al. (see Nucl. Acids Res. 16:3209 (1988)), and methyphosphonate oligonucleotides can be prepared by use of controlled pore glass polymer supports (see Sarin et al., Proc. Natl. Acad. Sci. USA 85:7448-51 (1988)), and the like.

In a specific embodiment, the FGF-19 antisense oligonucleotide comprises catalytic RNA, or a ribozyme (see, e.g., International Patent Publication WO 90/11364; Sarver et al., Science 247:1222-25 (1990)). In another embodiment, the oligonucleotide is a 2′-O-methylribonucleotide (see, e.g., Inoue et al., Nucl. Acids Res. 15:6131-48 (1987)), or a chimeric RNA-DNA analogue (see, e.g., Inoue et al., FEBS Lett. 215:327-30 (1987)).

In an alternative embodiment, the FGF-19 antisense nucleic acid of the invention is produced intracellularly by transcription from an exogenous sequence. For example, a vector can be introduced in vivo such that it is taken up by a cell, within which the vector or a portion thereof is transcribed, producing an antisense nucleic acid (RNA) of the invention. The vector would contain a sequence encoding the FGF-19 antisense nucleic acid or a portion thereof. Once inside the cell the vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art and used for replication and expression in mammalian cells. Expression of the sequence encoding the FGF-19 antisense RNA can be controlled by any promoter known in the art to act in mammalian, typically human, cells. The promoters can be inducible or constitutive. Inducible promoters include but are not limited to, the SV40 early promoter region (see Bemoist and Chambon, Nature 290:304-10 (1981)), the promoter contained in the 3′ long terminal repeat of Rous sarcoma virus (see Yamamoto et al., Cell 22:787-97(1980)), the herpes thymidine kinase promoter (see Wagner et al., Proc. Natl. Acad. Sci. USA 78:1441-45(1981)), the regulatory sequences of the metallothionein gene (see Brinster et al., Nature 296:39-42(1982)), and the like.

Animal Models:

The invention also provides animal models. In one embodiment, animal models for diseases and disorders involving angiogenesis are provided. Such an animal can be initially produced by promoting homologous recombination between an FGF-19 gene in its chromosome and an exogenous FGF-19 gene that has been rendered biologically inactive (typically by insertion of a heterologous sequence, such as an antibiotic resistance gene). For example, homologous recombination is carried out by transforming embryo-derived stem (ES) cells with a vector containing the insertionally inactivated FGF-19 gene, such that homologous recombination occurs, followed by injecting the ES cells into a blastocyst, and implanting the blastocyst into a foster mother, followed by the birth of the chimeric animal (“knockout animal”) in which an FGF-19 gene has been inactivated (see, e.g., Capecchi, Science 244:1288-1292 (1989); U.S. Pat. Nos. 5,631,153; 5,627,059; 5,487,992; and 5,464,764). The chimeric animal can be bred to produce additional knockout animals. Such animals can be mice, hamsters, sheep, pigs, cattle, and the like, and are typically non-human mammals. In a specific embodiment, a knockout mouse is produced. Knockout animals are expected to develop or be predisposed to developing diseases or disorders associated with hyper-angiogenic conditions and can be useful to screen for or test molecules for the ability to decrease angiogenesis and thus treat or prevent such diseases and disorders.

In another embodiment, transgenic animals that have incorporated and overexpress FGF-19 genes have use as animal models of diseases and disorders involving hypo-angiogenesis. Transgenic animals are expected to develop or be predisposed to hypo-angiogenic conditions, or exhibit increased resistance to diseases requiring angiogenesis, such as tumor formation. Thus, these animals can have use as animal models of such diseases and disorders, or the resistance to such diseases and conditions.

### EXAMPLES

The following examples are offered to illustrate, but no to limit the claimed invention.

### Example 1

Construction of Mammalian Expression Plasmid for Expressing FGF-19 in HEK-293 Cells.

The DNA sequence encoding the full length FGF-19 protein was amplified using PCR oligonucleotide primers corresponding to the 5′ and 3′ sequences of the gene. The clone was deposited at the ATCC (ATCC accession number 207014).

The expression vector pcDNA3 (InVitrogen) used for construction of the FGF-19 expression plasmid contains: 1) SV40 origin of replication, 2) ampicillin-resistance gene, 3) E. coli replication origin, 4) CMV promoter followed by a polylinker region and a BGH polyadenylation site, 5) SV40 promoter-Neo^{R}-SV40 polyadenylation site. DNA fragments encoding the entire FGF-19 precursor and an HA tag fused in-frame to the 3′ end is cloned into the polylinker region of the vector, therefore, the recombinant protein expression is under the control of CMV promoter. The HA tag (YPYDVPDYA) (Babco) corresponds to an epitope derived from the influenza hemagglutinin protein. The infusion of HA tag to the target protein allows easy detection of the recombinant protein with an antibody that recognizes the HA epitope (Babco).

The DNA sequence encoding human FGF-19, was amplified by PCR using two primers: the 5′ primer (5′CGGAAGCTTGCCATGCGGAGCGGGTGTGTGGTGG 3′ (SEQ ID NO:3)) contains a HindIII site followed by 25 nucleotides of coding sequence starting from 3 nucleotides before the initiation codon ATG; the 3′ primer (5′CGGTCTAGATTAGGCGTAATCTGGGACATCGTATGGGTACTTCTCAAAGCTGG GACTCCTCACGG 3′ (SEQ ID NO:4)) contains complementary sequences to an XbaI site, translation stop codon, HA tag and the last 26 nucleotides of the FGF-19 coding sequence (not including the stop codon). Therefore, the PCR product contains a HindIII site, coding sequence followed by in-frame HA tag, a stop codon next to the HA tag, and an XbaI site. The PCR amplified DNA fragment and the vector, pcDNA3, were digested with HindIII and XbaI (NEW ENGLAND BIOLAB), purified, and ligated. The ligation mixture was transformed into E. coli strain XL1-Blue (Stratagene) and the transformed culture was plated on LB/Amp plates. Plasmid DNA was isolated from transformants and examined by restriction analysis for the presence of the correct FGF-19 fragment and all the sequences were confirmed by sequencing. The expression plasmid was named pcDNA3-FGF-19-HA.

### Example 2

Transient Expression of Human FGF-19 in HEK-293 Cells.

The FGF-19 expression plasmid, pcDNA3-FGF-19-HA, was transiently transfected into HEK-293 cells (ATCC, Gaithersburg, Md.) using the method of lipofectinamine (Gibco-BRL). 48 hours after transfection, the cell pellet and supernatant media were boiled and denatured in sample buffer and subjected to SDS-PAGE (15%). After blotting the protein onto nitrocellulose paper, the recombinant FGF-19-HA was detected using a rabbit anti-HA antibody (Babco) with ECL detection (Amersham).

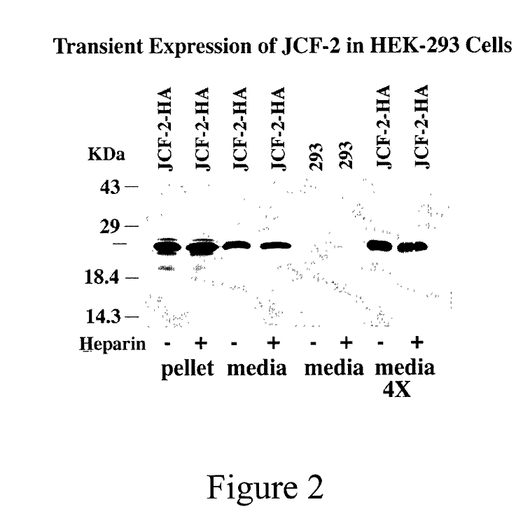

The coding sequence of FGF-19 plus the HA tag for detection of its expression by Western Blot are shown in FIG. 2. Molecular weight markers are highlighted on the left side of the gel. “293” indicates the mock-transfected HEK-293 cells. “Pellet” stands for cell pellet and “media” stands for supernatant collected from the cells. “4×” indicates the media were concentrated 4-fold using a Centriprep-10 (Amicon). “+/−heparin” in the legend means 100 ug/ml heparin were either added or not 12 hours before media were collected for analysis.

### Example 3

The Effect of FGF-19 Conditioned Medium on HUVEC Cells.

The FGF-19 gene was cloned into the LPSTGLNK vector by standard methods. This vector contains an EF1-alpha promoter which drives expression of a transgene, and an eGFP sequence which acts as an expression control. An IRES (internal ribosome entry site) sequence was used to coexpress JCF-2 with eGFP under the same promoter (Clontech, Palo Alto, Calif.). Plasmids LPSTGLNK-JCF2, and LPSTGLNK were transfected into HKB 11 cells using a transfection reagent called Fugene (Roche Biochemicals). The transfection was first conducted in 5% serum medium. 24 hours after transfection, the serum medium was replaced with defined medium. The culture supernatant was collected 96 hours after transfection. The supernatant was filtered through 0.22 um filter and then used for functional assays. Conditioned medium from LPSTGLNK transfected cells is referred to below as vector CM.

To test the effects of FGF-19 on angiogenesis, conditioned media was added to HUVEC cells plated on Matrigel. As a positive control, a mutant of IL8; namely, DLQ250, was added to previously untreated HUVECs as a purified protein at 250 nM. DLQ250 has been shown to inhibit capillary formation (Strieter, R. et. al. Jour Biol Chem 270(45) pp27348-27357 (1995)). The Matrigel coated plates were prepared as follows: Matrigel basement membrane matrix (Becton Dickinson) was thawed on ice overnight at 4° C. Pre-cooled pipettes, pipette tips, plates and tubes where used. 0.3 μl/well of Matrigel (at 4° C.) was used to coat the wells of a 24 well plate. The Matrigel was polymerized at 37° C. for 2 hours. Following addition of HUVEC to the Matrigel coated wells, the plates was incubated for 24 hours at 5% CO₂ and 37° C.

After 24 hours of incubation at 37° C., 5% CO₂, each well was checked under a microscope at low magnification (inverted microscope at ×10 power). The plates were then stained with Diff-Quick, and pictures were be taken to compare the controls with the different concentrations.

All tests were performed in triplicate wells. The final volume in each well is 1 ml of tissue culture medium alone or as modified in Table 1.

TABLE 1
Treatment of Cells in Matrigel

| Matrigel Treatment | Volume | Medium Volume |
|---|---|---|
| 1. HUVEC Control | 0 | 1 ml |
| 2. DLQ protein 250 nM | 0 | 1 ml |
| 3. Vector CM 1:10 | 100 μl | 900 μl |
| 4. Vector CM 1:100 | 10 μl | 990 μl |
| 5. FGF-19 CM 1:10 | 100 μl | 900 μl. |
| 6. FGF-19 CM 1:100 | 10 μl | 990 μl |

The results were as follows, where the length of the tubing formation is given in centimeters. (SEM indicates standard error of the mean).

TABLE 2
Endothelial tubule Formation

| | | | Vector CM | Vector CM | FGF-19 | FGF-19 |
|---|---|---|---|---|---|---|
| Condition | Control | DLQ 250 | 1:10 | 1:100 | CM 1:10 | CM 1:100 |
| Well 1 | 86.6 | 49.7 | 64.0 | 73.9 | 37.1 | 50.1 |
| Well 2 | 75.7 | 44.9 | 75.1 | 70.8 | 32.8 | 40.2 |
| Well 3 | 78.5 | 33.9 | 73.9 | 69.1 | 32.1 | 42.4 |
| Average | 80.3 | 42.8 | 71.0 | 71.3 | 34.0 | 44.2 |
| SEM | 5.7 | 8.1 | 6.1 | 2.4 | 2.7 | 5.2 |

As can be seen from this example, treatment of cells with tissue culture medium containing FGF-19 significantly reduces the extent of capillary tubule formation. The extent of reduction is equivalent to reduction seen with DLQ250. Treatment with medium from cells transfected with the LPSTGL vector did not result in inhibition of tubule formation. Thus, this experiment demonstrates that FGF-19 polypeptide inhibits angiogenesis.

### Example 4

The Effect of FGF-19 Gene Expression on Endothelial Tube Formation.

The effect of FGF-19 gene expression on endothelial tube formation was further examined by studying HUVEC capillary like-organization in Matrigel following adenoviral based gene delivery of FGF-19.

Adenovirus expression vectors containing FGF-19 cDNA were constructed by excising FGF-19 cDNA from pcDNA3.1/FGF-19/HA by digestion with Eco RV and Pmel. The resulting fragment was cloned into the corresponding sites of pShuttle-CMV (He et al., Proc. Nat. Acad. Sci. USA 95:2509-14 (1998)). pShuttle-CMV is an adenovirus shuttle vector in which the transgene (i.e., FGF-19 cDNA) is under the control of the cytomegalovirus (“CMV”) promoter. This construct was transferred to an adenoviral backbone by recombination in E. coli with the plasmid pAdEasy (“AdEZ”) (He et al., supra). The resulting recombinant was linearized with the restriction endonuclease Pac 1 and then transfected into HEK293 cells (Microbix Inc, Ontario, Canada) by standard methods. Ten days post-transfection (after the appearance of viral plaques), vector particles were harvested from the cells by multiple rounds of freeze-thawing. Particles were then used to infect 911 epithelial cells (Introgene, Leiden, Netherlands). To obtain sufficient vector for multiple experiments, three rounds of passage of viral particles to successively larger cultures of 911 cells were performed. This viral stock was termed “Ad-FGF-19.” This crude stock was used in the following experiment.

HUVEC were plated at 1.25×10⁴ cells per well at day zero in a 24 well plate. The cells were infected with Ad-FGF-19 stock, with the control vector Ad-Ez, Ad-VEGF stock (Vascular Endothelial Growth Factor). 200 μl of each viral stock was added to the wells on day 0. As a further control, lysates from the 911 cell line, which is used to grow the Ad vectors, was added to a separate set of wells. Plates were incubated in 5% CO₂ at 37° C. for 5 days. On day 6, the cells were passed to a 24 well plate coated with Matrigel at a density of 3×10⁴ cells/well. As a positive control, DLQ250, the mutant of IL8 described in Example 3, was added to previously untreated HUVECs as a purified protein at 250 nM. The Matrigel coated plates were prepared as follows: Matrigel basement membrane matrix (Becton Dickinson) was thawed on ice overnight at 4° C.. Pre-cooled pipettes, pipette tips, plates and tubes where used. 0.3 μl/well of Matrigel (at 4° C.) was used to coat the wells of a 24 well plate. The Matrigel was polymerized at 37° C. for 2 hours. Following addition of HUVEC to the Matrigel coated wells, the plates was incubated for 24 hours at 5% CO₂ and 37° C. All tests were performed in triplicate wells. The final volume in each well is 1 ml.

After 24 hours of incubation at 37° C., 5% CO₂, each well was checked under a microscope at low magnification (inverted microscope at ×10 power). The plates were then stained with Diff-Quick, and pictures were be taken to compare the controls with the different concentrations.

The following tables summarize the protocol.

TABLE 3
Treatment of HUVECs with adenoviral vectors

| No Matrigel treatment (5 days) | Volume | Medium Volume |
|---|---|---|
| 1. HUVEC Control (6 wells) | 0 | 1 ml. |
| 2. 911 Sup. plus cell Lysate | 200 μl. | 800 μl. |
| 3. Ad-EZ plus cell Lysate | 200 μl | 800 μl |
| 4. Ad-VEGF plus cell Lysate | 200 μl. | 800 μl. |
| 5. Ad-FGF-19 plus cell Lysate | 200 μl. | 800 μl. |

The results were as follows, where the length of the tubing formation is given in centimeters. (SEM indicates standard error of the mean.)

TABLE 4
Endothelial Tubing Formation

| Condi- | Con- | DLQ | | | | |
|---|---|---|---|---|---|---|
| tion | trol | 250 | 911 Lys. | Ad-EZ | Ad-VEGF | Ad-FGF-19 |
| Well 1 | 68.22 | 24.06 | 82 | 60.8 | 78.6 | 17.2 |
| Well 2 | 81.4 | 16.12 | 88.8 | 63.2 | 69.6 | 22 |
| Well 3 | 70.56 | 22.5 | 75.2 | 89.4 | 68.8 | 19.8 |
| Avg. | 73.39 | 20.89 | 82.00 | 71.13 | 72.33 | 19.67 |
| SEM | 4.06 | 2.43 | 4.61 | 9.16 | 3.14 | 1.39 |

As can be seen from this example, the control cells and cells infected with the vector alone (Ad-EZ) showed similar amounts of tubing formation. Expression of VEGF also produced similar levels of tubing formation. In contrast, expression of FGF-19 polypeptide markedly inhibited tubing formation. Thus, this experiment demonstrates that FGF-19 polypeptide, expressed from an adenoviral vector, inhibits angiogenesis.

### Example 5

The Effect of ex vivo Transduction of B16 Murine Melanoma Cells with FGF-19 on in vivo Tumor Metastasis.

The effect of in vivo expression of FGF-19 in B16 murine melanoma metastasis was examined. Briefly, the FGF-19 cDNA was delivered ex vivo with an adenoviral vector (Ad-FGF-19) followed by introduction of the transfected cells into mice. 74 females C 57 B1/6, 6-8 weeks old, were used. For ex vivo administration, either crude lysates (prepared as described in Example 3) or purified vectors were used. The adenoviral vectors were purified as follows: The protocol is a modification of Fallux et al. (Human Gene Therapy 7:215-22 (1996)). For large scale purification, 911 cells were plated in a Multi-tray Cell Factory (Nuclon, Denmark). When the cells reached 85% confluence, they were infected with the recombinant adenovirus. Following infection, about 48-72 hours post-infection, when the cells showed a cytopathic effect, the cells were harvested and centrifuged. The cell pellet was resuspended in a small volume of medium, three cycles of freezing and thawing were performed, and the disrupted cells were pelleted to remove the cellular debris. The viral particle-containing supernatants were layered onto a discontinuous cesium chloride (CsCl) gradient composed of 1 ml at d=1.4 g/ml overlaid with 3 mls of d=1.25 g/ml. The gradients were centrifuged at 151,000×g for 2 hours. The opaque band of virus particles, at the 1.25/1.4 density boundary, was collected and loaded onto a homogeneous CsCl solution of d=1.3 g/ml. This second gradient was spun at 151,000×g for 18 hours. The single band of virus particles was collected and dialyzed twice for one hour against 0.135M NaCl, 1 mM MgCl₂, 10 mM Tris pH 7.5. The second and final dialysis was carried out against the same buffer with the addition of 10% glycerol. Stock titers were determined by plaque assay using 293 or 911 cells.

B16.F10 cells, from a murine melanoma metastasis were infected for 24 hours with one of the following adenovirus expression vectors: Ad-El (control), Ad-FGF-19 or Ad-VEGF. The infected cells were injected intravenously into the lateral tail vein of the mice at the end of the 24 incubation period. The cell concentration of each injection was 2×10⁵ cells in 0.2 ml of PBS. Day 0 was the date of injection into mice.

The animals were weighed two times a week. Two animals from group 1 (control) were sacrificed on day 14, the lungs collected and the number of metastases determined. Following counting of the metastases, the remainder of the animals (10 in each group) were sacrificed on day 14. At that time, lungs were collected, weighed and the number of metastases counted.

The following table summarizes the experimental protocol.

TABLE 5

| | NUMBER OF | | |
|---|---|---|---|
| GROUP | ANIMALS | CELL INFECTION | SACRIFICED ON |
| 1 | 10 | None | Day 14 (only 2 mice), |
| 2 | 10 | AdEZ | Day 14 |
| 6 | 9 | Ad-VEGF | Day 14 |
| 8 | 9 | Ad-FGF-19 | Day 14 |

The following Table 6 summarizes the lung weights from each group. SEM is the “standard error of the mean.”

TABLE 6
Lung Weight

| Group | Average | SEM | Median |
|---|---|---|---|
| 1 (control) | 180.3 | 10.4 | 194.3 |
| 2 (AdEz) | 376.4 | 34 | 362.3 |
| 6 (Ad-VEGF) | 265.3 | 21.9 | 260.4 |
| 8 (Ad-FGF-19) | 245.5 | 19.9 | 238.1 |

The following Table 7 summarizes the number of lung metastases from each group. SEM is the “standard error of the mean.”

TABLE 7
Lung Metastases

| Group | Average | SEM | Median |
|---|---|---|---|
| 1 (control) | 54 | 4.8 | 54 |
| 2 (AdEZ) | 200 | 0 | 200 |
| 6 (Ad-VEGF) | 66 | 23 | 43 |
| 8 (Ad-FGF-19) | 5.7 | 1.5 | 4 |

As can be seen from this data, the average lung weight in mice receiving tumor cells overexpressing FGF-19 cDNA was markedly lower than in control animals. The average lung weight in AD-VEGF and Ad-FGF-19 treated animals was similar. More importantly, and referring to Table 6, the average number of lung metastases was over 10 times less in tumor cells overexpressing FGF-19 polypeptide as compared with control and VEGF-treated animals. Thus, this data reveal that overexpression of FGF-19 cDNA inhibits tumor cell growth. The increase in the number of tumors in the Ad-EZ controls is unexpected and currently under investigation.

### Example 6

The Effect of in vivo Expression of FGF-19 on B16 Murine Melanoma Metastasis.

The effect of in vivo expression of FGF-19 in B16 murine melanoma metastasis model was examined. Briefly, 2×10e5 B16.F10 cells, in 0.2 ml PBS, were injected into the lateral tail vein of 6-8 week-old female SCID mice. One day later, the mice were injected i.v. with either control adenovirus vector (CMV-Null) or vector containing the FGF-19 cDNA constituting a dose of 1×10e9 pfu/mouse in 0.2 ml PBS. Adenoviral vector construction and purification were as described in examples 4 and 5 with minor modifications. The shuttle plasmid pAdTrack was digested with HindIII and XbaI and the FGF-19 cDNA fragment from pcDNA3.1/FGF-19/HA was inserted into these sites. In this construct, FGF-19 is also under control of the CMV promoter. (He et al., Proc. Nat. Acad. Sci. USA 95:2509-14 (1998). Fourteen days later, the animals were sacrificed, the lungs collected and the metastases were counted under a dissecting microscope.

The following Table 8 summarizes the number of lung metastases from each group. SEM is the “standard error of the mean”.

TABLE 8
Lung Metastases

| Group | Average | SEM | Median |
|---|---|---|---|
| Control | 128 | 14 | 120 |
| (No viral injection) | | | |
| CMV-Null | 136 | 22 | 156 |
| (Empty vector) | | | |
| Ad-FGF-19 | 32 | 6 | 31 |

As can be seen from the data, the average number of metastases detected in the lungs of mice receiving Ad-FGF-19 was lower than in untreated animals or those receiving the empty viral vector. These data demonstrate that in vivo expression of FGF-19 protein inhibits tumor cell growth in this model.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for inhibiting angiogenesis comprising administering to a mammal a therapeutically effective dose of an isolated FGF-19 polypeptide, a fragment, variant, derivative or analog thereof.

2. The method of claim 1, wherein the FGF-19 polypeptide is human FGF-19 (SEQ ID NO:2).

3. The method of claim 1, wherein the mammal is human.

4. The method of claim 1, wherein the administering is performed in vivo.

5. The method of claim 1, further comprising administering a pharmaceutical carrier.

6. The method of claim 1, wherein the administering is ex vivo.

7. The method of claim 6, further comprising preparing a recombinant cell transfected with an FGF-19 nucleic acid and administering the cell to the mammal.

8. The method of claim 1, wherein the FGF-19 polypeptide is recombinantly expressed.

9. The method of claim 8, wherein the FGF-19 polypeptide is recombinantly expressed by culturing a cell containing an FGF-19 nucleic acid under conditions which result in expression of the polypeptide, and recovering the FGF-19 polypeptide from the cell culture.

10. The method of claim 8, wherein the FGF-19 polypeptide is expressed in E. coli.

11. The method of claim 8, wherein the FGF-19 polypeptide is expressed mammalian cells.

12. The method of claim 9, wherein the FGF-19 nucleic acid is operably linked to a promoter in an expression vector.

13. The method of claim 9, wherein the FGF-19 nucleic acid has the sequence of human FGF-19 (SEQ ID NO:1).

14. A method of treating a patient in need of anti-angiogenesis therapy comprising administering to the patient a therapeutically effective amount of an isolated FGF-19 polypeptide, fragment, variant, derivative or analog thereof.

15. A method of treating a disease in a subject in need of anti-angiogenesis therapy comprising administering to a subject a therapeutically effective amount of an FGF-19 polypeptide, fragment, variant, derivative or analog thereof.

16. The method of claim 15 wherein the disease is a tumor.

17. The method of claim 15, wherein the FGF-19 polypeptide is human FGF-19 (SEQ ID NO:2).

18. The method of claim 15, wherein the subject is human.

19. The method of claim 15, wherein the administering is performed in vivo.

20. The method of claim 15, further comprising administering a pharmaceutical carrier.

21. The method of claim 15, wherein the administration is ex vivo.

22. The method of claim 21, further comprising preparing a recombinant cell transfected with an FGF-19 nucleic acid and administering the cell to the subject.

23. The method of claim 15, wherein the FGF-19 polypeptide is recombinantly expressed.

24. The method of claim 23, wherein the FGF-19 polypeptide is recombinantly expressed by culturing a cell containing an FGF-19 nucleic acid under conditions which result in expression of the polypeptide, and recovering the FGF-19 polypeptide from the cell culture.

25. The method of claim 23, wherein the FGF-19 polypeptide is expressed in a cell, said cell is selected from the group consisting of mammalian cells, yeast cells, bacterial cells, and insect cells.

26. The method of claim 23, wherein the FGF-19 polypeptide is expressed mammalian cells.

27. The method of claim 24, wherein the FGF-19 nucleic acid is operably linked to a promoter in an expression vector.

28. The method of claim 24, wherein the expression vector is an adenoviral vector, a retroviral vector, or a lentiviral vector.

29. The method of claim 24, wherein the FGF-19 nucleic acid has the sequence of human FGF-19 (SEQ ID NO:1).

30. A method of inhibiting endothelial tube formation comprising administering to an endothelial cell an effective amount of an FGF-19 polypeptide, or fragment, variant, derivative, analog or a pharmaceutically acceptable salt thereof.

31. The method of claim 30, wherein the FGF-19 polypeptide is human FGF-19.

32. A method of inhibiting tumor cell growth comprising administering to a tumor cell an effective amount of an FGF-19 polypeptide, or fragment, variant, derivative, analog or a pharmaceutically acceptable salt thereof.

33. The method of claim 32, wherein the FGF-19 polypeptide is human FGF-19.

34. A method of treating a disease in a subject in need of anti-angiogenesis therapy comprising administering to a subject a nucleic acid sequence encoding an FGF-19 polypeptide, fragment, variant, derivative or analog thereof.

35 The method of claim 34 where in the FGF-19 polypeptide has the sequence of human FGF-19 (SEQ ID NO:2).
